(19) United States
(12) Patent Application Publication
(10) Pub. No.: US 2009/0275502 A1
(43) Pub. Date: Nov. 5, 2009

(54) METHODS AND COMPOSITIONS FOR ENHANCING DEVELOPMENTAL POTENTIAL OF OOCYTES AND PREIMPLANTATION EMBRYOS

(75) Inventors: Andrea Jurisicova, Richmond Hill (CA); Robert Casper, Toronto (CA); Gloria I. Perez, East Lansing, MI (US)

Correspondence Address: HOWSON & HOWSON LLP, 501 OFFICE CENTER DRIVE, SUITE 210, FORT WASHINGTON, PA, 19034, US

(73) Assignees: MOUNT SINAI HOSPITAL, Toronto, ON (CA); GENERAL HOSPITAL CORPORATION, Charlestown, MA (US); BOARD OF TRUSTEES OF MICHIGAN STATE UNIVERSITY, East Lansing, MI (US)

(21) Appl. No.: 11/917,675

(22) PCT Filed: Jun. 16, 2006
(86) PCT No.: PCT/CA2006/000991

§ 371 Date: Jan. 23, 2009

Related U.S. Application Data
(60) Provisional application No. 60/692,011, filed on Jun. 17, 2005.

(51) Int. Cl. A61K 38/17; A61B 17/435; C12N 5/06; A61K 38/02; C12N 13/00

(52) U.S. Cl. 514/12; 600/34; 435/375; 514/2; 435/173.1

(57) ABSTRACT

The invention relates to compositions and methods for enhancing the developmental potential of oocytes or preimplantation embryos by modulating mitochondrial-associated proteins and/or genomic integrity modifier proteins in the oocytes or preimplantation embryos. In one aspect of the invention, the levels of one or more mitochondrial-associated proteins and/or genomic integrity modifier proteins are increased, in particular by introducing the mitochondrial-associated proteins and/or genomic integrity modifier proteins into the oocytes or preimplantation embryos. Oocytes may be fertilized to obtain a zygote with increased levels of one or more mitochondrial-associated proteins and/or genomic integrity modifier proteins. The methods and compositions may be used to improve in vitro fertilization and embryo transfer methods, and nuclear transfer techniques.

### FIELD OF THE INVENTION

The invention relates to compositions and methods for enhancing the developmental potential of oocytes and preimplantation embryos.

### BACKGROUND OF THE INVENTION

Mammalian preimplantation embryo development is prone to high rates of early embryo wastage, particularly under current in vitro culture conditions. There are many possible underlying causes for embryo demise including DNA damage, altered embryo metabolism and the effect of suboptimal culture media, all of which contribute to an imbalance in gene expression and the failed execution of basic embryonic decisions. An increasing body of evidence indicates that cell fate is determined by the outcome of specific intracellular interactions between pro- and anti-apoptotic proteins, many of which are expressed during oocyte and preimplantation embryo development.

### SUMMARY

The present invention relates to a method for enhancing developmental potential of oocytes and preimplantation embryos comprising modulating in the oocytes or preimplantation embryos modifiers of genetic integrity and/or mitochondrial ultrastructure that influence apoptosis.

The present invention also relates to a method for enhancing developmental potential of oocytes and preimplantation embryos comprising administering an effective amount of modifiers of genetic integrity and/or mitochondrial ultrastructure that influence apoptosis to reverse, decrease, or inhibit reduced DNA repair capacity (e.g., defective DNA repair), DNA damage, mitochondrial defects and/or deficiencies in reactive oxygen species (ROS) production. Modifiers of genetic integrity and/or mitochondrial ultrastructure that influence apoptosis include mitochondrial-associated proteins (in particular Bcl-2 family proteins) and/or genomic integrity modifier proteins (in particular DNA repair proteins).

The invention further relates to a method for decreasing, inhibiting or reversing reduced DNA repair capacity (e.g. defective DNA repair), DNA damage, mitochondrial defects, and/or deficiencies in ROS production in oocytes or preimplantation embryos comprising administering to the oocytes or preimplantation embryos one or more mitochondrial-associated proteins (in particular Bcl-2 family proteins) and/or genomic integrity modifier proteins (in particular DNA repair proteins).

The invention relates to a method for enhancing developmental potential of oocytes and preimplantation embryos comprising modulating mitochondrial-associated proteins (in particular Bcl-2 family proteins), and/or modulating genomic integrity modifier proteins (in particular DNA repair proteins) in the oocytes and preimplantation embryos. Mitochondrial-associated proteins and genomic integrity modifier proteins may be modulated by introducing the proteins into the oocytes or preimplantation embryos.

In aspects of the invention, the developmental potential of oocytes is enhanced by modulating one or more Bcl-2 family proteins, in particular pro-survival Bcl-2 family proteins. In some aspects, levels of pro-survival Bcl-2 family proteins are increased in the oocytes. In an embodiment of the invention, the levels of pro-survival Bcl-2 family proteins are increased by introducing one or more pro-survival Bcl-2 family proteins or agonists thereof into the oocytes. A method of the invention may additionally comprise fertilizing the oocytes to obtain a zygote with increased levels of one or more pro-survival Bcl-2 family proteins.

In other aspects of the invention, the developmental potential of oocytes is enhanced by modulating one or more RecA family proteins, in particular Rad51 family proteins. In some aspects, levels of RecA family proteins, in particular Rad51 family proteins are increased in the oocytes. In an embodiment of the invention, the levels of RecA family proteins, in particular Rad51 family proteins, are increased by introducing one or more RecA family proteins, in particular Rad51 family proteins, or agonist thereof into the oocytes. A method of the invention may additionally comprise fertilizing the oocytes to obtain a zygote with increased levels of one or more RecA family proteins, in particular Rad 51 family proteins.

The invention also relates to a method for enhancing developmental potential of preimplantation embryos comprising modulating mitochondrial-associated proteins (in particular Bcl-2 family proteins) or modulating genomic integrity modifier proteins (in particular DNA repair proteins) in the preimplantation embryos. In an aspect, the proteins are modulated by increasing levels of one or more Bcl-2 family proteins and/or RecA family proteins in the preimplantation embryos. In an embodiment of the invention, the levels of pro-survival Bcl-2 family proteins are increased by introducing one or more pro-survival Bcl-2 family proteins or agonist thereof into the preimplantation embryos. In another embodiment of the invention, the levels of Rad51 family proteins are increased by introducing one or more Rad51 family proteins (e.g., Rad51) or an agonist thereof into the preimplantation embryos.

In an embodiment, the preimplantation embryo is a zygote and one or more mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins) are introduced in the zygote. In a particular embodiment, the proteins are introduced into the pronucleus or cytoplasm.

The invention further relates to an oocyte or a preimplantation embryo wherein one or more mitochondrial-associated proteins (in particular Bcl-2 family proteins) and/or genomic integrity modifier proteins (in particular DNA repair proteins, more particularly RecA family proteins, most particularly Rad51 family proteins), in the oocyte or preimplantation embryo are modulated. In an aspect, an oocyte or a preimplantation embryo obtained from a method of the invention is provided wherein the oocyte or preimplantation embryo comprises increased levels of one or more pro-survival Bcl-2 family proteins, in particular pro-survival Bcl-2 family proteins. In another aspect, an oocyte or a preimplantation embryo obtained from a method of the invention is provided wherein the oocyte or preimplantation embryo comprises increased levels of one or more DNA repair proteins, in particular RecA family proteins, more particularly Rad51 family proteins.

In a further aspect the invention relates to a composition comprising at least one, two, three, four or more Bcl-2 family proteins (in particular pro-survival Bcl-2 family proteins) and/or genomic integrity modifier proteins (in particular DNA repair proteins, more particularly RecA family proteins, most particularly Rad51 family proteins), in a form or effective amount for enhancing developmental potential of oocytes or preimplantation embryos. In an embodiment a composition of the invention comprises one or more of Bcl-2, Bcl-xL, Mcl-1, Diva, and Aven, in particular Bcl-2, Bcl-xL, and Mcl-1, and a pharmaceutically acceptable carrier, excipient or vehicle. In a still further embodiment a composition of the invention comprises one or more of RecA family proteins, in particular Rad51 family proteins, more particularly Rad51, and a pharmaceutically acceptable carrier, excipient or vehicle. In another embodiment, a composition of the invention comprises Bcl-xΔC that lacks the C terminus, or Bcl-xES lacking the BH3/BH1 region [Schmitt, 2004], and a pharmaceutically acceptable carrier, excipient, or vehicle. In a particular embodiment, a composition of the invention comprises a pro-survival Bcl-2 family protein and/or Rad51 family protein with a terminal half-life of less than about 24, 20, 15, 10, 9, 8, 7, 6, or 5 hours.

The invention relates to the use of one or more mitochondrial-associated proteins (in particular Bcl-2 family proteins, more particularly pro-survival Bcl-2 family proteins) and genomic integrity modifier proteins (in particular DNA repair proteins, more particularly RecA family proteins, most particularly Rad51 family proteins), in the manufacture of a medicament for use in improving embryo development after in vitro fertilization or embryo transfer in a female mammal.

The invention also relates to the use of one or more mitochondrial-associated proteins (in particular Bcl-2 family proteins, more particularly pro-survival Bcl-2 family proteins) and genomic integrity modifier proteins (in particular DNA repair proteins, more particularly RecA family proteins, most particularly Rad51 family proteins) in the manufacture of a medicament for use in reducing, inhibiting or decreasing reduced DNA repair capacity, DNA damage, mitochondrial defects, and/or deficiencies in ROS production in oocytes or preimplantation embryos.

In another aspect, the invention provides a method for fertilizing oocytes comprising removing oocytes from a follicle of an ovary, modulating mitochondrial-associated proteins (in particular Bcl-2 family proteins) and/or genomic integrity modifier proteins (in particular DNA repair proteins, more particularly RecA family proteins, most particularly Rad51 proteins) in the oocytes, and fertilizing the resulting oocytes with spermatozoa.

In another aspect, the invention provides a method for fertilizing oocytes comprising removing oocytes from a follicle of an ovary, introducing one or more mitochondrial-associated proteins (in particular Bcl-2 family proteins, more particularly pro-survival Bcl-2 family proteins) and/or genomic integrity modifier proteins (in particular DNA repair proteins, more particularly RecA family protein, most particularly Rad51 family proteins) into the oocytes, and fertilizing the resulting oocytes with spermatozoa. The introduction of the proteins and the spermatozoa can be carried out simultaneously, sequentially, or separately. In an embodiment, the proteins and spermatozoa are simultaneously injected.

In a still further aspect the invention provides a method for storing and then enhancing the developmental potential of oocytes comprising cryopreserving immature oocytes, thawing the cryopreserved oocytes, and modulating mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins), in the oocytes.

In a still further aspect the invention provides a method for storing and then enhancing the developmental potential of oocytes comprising cryopreserving immature oocytes, thawing the cryopreserved oocytes, and introducing one or more mitochondrial-associated protein, in particular Bcl-2 family proteins, more particularly pro-survival Bcl-2 family proteins, and/or genomic integrity modifier proteins (in particular DNA repair proteins, more particularly RecA family protein, most particularly Rad51 family proteins), into the oocytes.

The methods and compositions of the invention can improve the quality of the oocytes that are being fertilized and the quality of preimplantation embryos to increase the rate of success in embryo development and ongoing pregnancy.

In an aspect, the invention provides a method for improving embryo development after in vitro fertilization or embryo transfer in a female mammal comprising implanting into the female mammal an embryo derived from an ooctye or preimplantation embryo (e.g., zygote), wherein one or more mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins) have been modulated.

In an aspect, the invention provides a method for improving embryo development after in vitro fertilization or embryo transfer in a female mammal comprising implanting into the female mammal an embryo derived from an ooctye or preimplantation embryo (e.g., zygote) comprising increased levels of one or more pro-survival Bcl-2 family proteins and/or Rad51 family proteins.

The invention provides methods of improving the success of in vitro fertilization, gamete intrafallopian transfer, or zygote intrafallopian transfer comprising modulating one or more mitochondrial-associated protein (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins) in oocytes or preimplantation embryos employed therein.

In an aspect, the invention provides a method for improving the success of in vitro fertilization in a female subject comprising:

(a) removing oocytes from the subject;

(b) modulating one or more mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins) in the oocytes;

(c) fertilizing the oocytes with spermatozoa; and

(d) transferring fertilized oocytes from step (c) into the uterus of the subject.

In another aspect, the invention provides a method for improving the success of zygote intrafallopian transfer in a female subject comprising:

(a) removing oocytes from the subject;

(b) modulating one or more mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins) in the oocytes;

(c) fertilizing the oocytes with spermatozoa; and

(d) transferring fertilized oocytes from step (c) into a fallopian tube of the subject.

In a further aspect, the invention provides a method for improving the success of gamete intrafallopian transfer in a female subject comprising:

(a) removing oocytes from the subject;

(b) modulating one or more mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins) in the oocytes;

(c) combining the oocytes with spermatozoa; and

(d) immediately introducing the oocytes and spermatozoa from step (c) into a fallopian tube of the subject.

In the above methods, mitochondrial-associated proteins (in particular Bcl-2 family proteins, more particularly pro-survival Bcl-2 family proteins), and/or genomic integrity modifier proteins (in particular DNA repair proteins, more particularly RecA family proteins, most particularly Rad51 family proteins) may be modulated in the oocytes by introducing the proteins or agonists thereof into the oocytes.

An oocyte may be a recipient oocyte in a nuclear transfer method. Thus, the invention relates to a method for enhancing developmental potential of recipient oocytes in a nuclear transfer method comprising modulating mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins), in the recipient oocytes. In particular, the invention relates to a method for enhancing developmental potential of recipient oocytes in a nuclear transfer method comprising introducing one or more mitochondrial-associated proteins, in particular Bcl-2 family proteins, more particularly pro-survival Bcl-2 family proteins, into the recipient oocytes. In another aspect, the invention relates to a method for enhancing developmental potential of recipient oocytes in a nuclear transfer method comprising introducing one or more genomic integrity modifier proteins (in particular DNA repair proteins, more particularly RecA family proteins, most particularly Rad51 family proteins), into the recipient oocytes.

The invention also contemplates recipient oocytes comprising exogenous (e.g., isolated or recombinant) mitochondrial-associated proteins, in particular Bcl-2 family proteins, more particularly pro-survival Bcl-2 family proteins, and/or genomic integrity modifier proteins (in particular DNA repair proteins, more particularly RecA family proteins, most particularly Rad51 family proteins) and preimplantation embryos, blastocyts, embryos, and non-human animals formed from a nuclear transfer method of the invention. In conventional nuclear transfer methods, the donor nucleus is placed in an enucleated oocyte obtained from a different individual. The invention by introducing mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins) into recipient oocytes enhances the developmental potential of the recipient oocytes. This is expected to increase the live birth rate in nuclear transfer methods.

In an embodiment, the invention provides a method of cloning a non-human mammalian embryo by nuclear transfer comprising:

(a) introducing a donor cell nucleus derived from a donor cell of a non-human mammal and exogenous mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins), preferably from the same species as the donor cell, more preferably from the same species and cell type as the donor cell, most preferably from the non-human mammal from which the donor cell nucleus is derived, into an enucleated recipient oocyte of the same species as the donor cell to form a nuclear transfer unit; and

(b) culturing the nuclear transfer unit to form an embryo.

The method may further comprise permitting the embryo to develop into a cloned mammal.

The invention also provides a method of cloning a non-human mammal by nuclear transfer comprising:

(a) introducing a donor cell nucleus derived from a donor cell of a non-human mammal, and exogenous mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins) from the same species as the donor cell, more preferably from the same species and cell type as the donor cell, most preferably from the non-human mammal from which the donor cell nucleus is derived, into a non-human mammalian enucleated recipient oocyte of the same species as the donor cell to form a nuclear transfer unit,

(b) culturing the nuclear transfer unit to form an embryo;

(c) implanting the embryo into the uterus of a surrogate mother of said species; and

(d) permitting the embryo to develop into the cloned mammal.

In yet another embodiment, a method of cloning a non-human mammalian fetus by nuclear transfer is provided comprising the following steps:

(a) introducing a donor cell nucleus from a donor cell of a non-human mammal, and mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins), preferably from the same species as the donor cell, more preferably from the same species and cell type as the donor cell, most preferably from the non-human mammal from which the donor cell nucleus is derived, into an enucleated recipient oocyte of the same species as the donor cell to form a nuclear transfer unit;

(b) culturing the nuclear transfer unit until greater than the 2-cell developmental stage; and

(c) transferring the cultured nuclear transfer unit to a host non-human mammal of the same species such that the nuclear transfer unit develops into a fetus.

The method may also comprise developing the fetus into an offspring.

In a further aspect the invention provides a recipient oocyte comprising a perivitelline space and a donor cell nucleus and mitochondrial-associated proteins (in particular Bcl-2 family proteins, more particularly pro-survival Bcl-2 family proteins) and/or genomic integrity modifier proteins (in particular DNA repair proteins, more particularly RecA family proteins, most particularly Rad51 family proteins), preferably from the same species as the donor cell, more preferably from the same species and cell type as the donor cell, most preferably from the same individual from which the donor cell nucleus is derived, deposited in the perivitelline space.

The invention also includes kits and articles-of-manufacture for conducting the methods of the invention.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the effect of culture conditions on expression of Bcl-x protein in ICR embryos. Expression of Bcl-x was determined by immunocytochemistry followed by computer aided decon analysis. ˜25% reduction in the intensity of fluorescence was observed in embryos cultured in the HTF medium, indicating association between developmental competence and Bcl-x expression.

FIG. 2 shows the effect of culture conditions on the expression of Mcl-1 in ICR embryos. (A.) Expression of Mcl-1 transcript was evaluated by quantitative RT-PCR followed by dot blot southern blot. (B.) Mcl-1 protein, as determined by immunocytochemistry followed by computer aided decon analysis (C.) ˜15% reduction in protein was detected in the HTF medium, followed by additional 10% increase in the reduction in the arrested 2-cell embryos.

FIG. 3 shows the effect of recombinant Bcl-x protein injection on blastocyst formation. Nuclear staining of untreated ICR embryos cultured in HTF medium (A), buffer injection (B) and recombinant Bcl-x protein injected (C,D.) Rates of blastocyst formation (E) of ICR zygotes cultured in the HTF medium without microinjection, with microinjection of buffer or recombinant Bcl-x protein (1.5 mg/ml) into pro-nucleus. Cell number and cell death rates (F) determined by nuclear morphology and TUNEL labeling in the obtained embryos.

FIG. 4 shows that the genetic background alters germline apoptosis susceptibility. Percentage of oocytes collected from adult B6C3F1, FVB and AKR/J female mice that exhibited apoptosis after a 24 h culture. Values are the mean ±SEM of combined data from analyzing the total number of oocytes of each strain indicated over the respective bar (asterisks, P <0.05 versus B6C3F1).

FIG. 5 shows defective DNA repair enhances apoptosis susceptibility in the AKR/J background. (A-C) Comet assay analysis of DNA integrity in freshly isolated B6C3F1, FVB or AKR/J oocytes. (D) Percent of undamaged (open bars) and damaged (filled bars) DNA in freshly isolated oocytes of the three indicated genetic strains. Values are the mean ±SEM of combined data from an analysis of 56-84 oocytes per strain as indicated over each bar. (E) Microinjection of recombinant Rad51 protein (+Rad51) reduces the percent of damaged DNA in AKR/J oocytes following a 6-h incubation. Values are the mean ±SEM of combined data from analyzing the total number of oocytes indicated per group over each bar (—Rad51, noninjected AKR/J oocytes; asterisk, P <0.05 versus noninjected group). (F) Microinjection of recombinant Rad51 suppresses apoptosis in AKR/J oocytes but has no effect on the high incidence of apoptosis in FVB oocytes studied in parallel. Results from analysis of non-injected (Control) and BSA-injected oocytes of each strain are also provided. Values are the mean ±SEM of combined data from analyzing the total number of oocytes indicated per group over each bar (asterisk, P <0.05 versus all other groups).

FIG. 6 shows Rad51 rescues the compromised developmental competence of female AKR/J germ cells. (A) In-vitro fertilization rates of B6C3F1, C57BL/6 and AKR/J oocytes. Values are the mean ±SEM of combined data from analyzing the total number of oocytes indicated per group over each bar (different letters, P <0.05). (B) Preimplantation embryonic developmental competence of B6C3F1, C57BL/6 and AKR/J zygotes. Microinjection of recombinant Rad51 protein into AKR/J zygotes increases blastocyst formation rates to levels not different from those observed with C57BL/6 zygotes. Values are the mean ±SEM of combined data from analyzing the total number of zygotes indicated per group over each bar (different letters, P <0.05).

FIG. 7 shows pyruvate reverses impaired ROS output and prevents apoptosis in FVB germ cells. (A) Freshly isolated FVB oocytes possess extremely low levels of ROS compared to oocytes of the other strains, indicative of reduced mitochondrial metabolic function. Values are the mean ±SEM of combined data from analyzing the total number of oocytes of each strain as indicated over the respective bar (asterisk, P <0.05 versus B6C3F1 or AKR/J). (B) Incubation of FVB oocytes in the presence of 10 mM pyruvate for 6 h increases ROS content to levels approaching those observed in freshly isolated B6C3F1 oocytes (see 7A). Values are the mean ±SEM of combined data from analyzing the total number of oocytes indicated per group over each bar. (C) Pyruvate completely reverses the high apoptosis susceptibility observed in vehicle-exposed FVB oocytes cultured for 24 h in parallel. Values are the mean ±SEM of combined data from analysis of the total number of oocytes indicated per group over each bar (N.D., none detected).

FIG. 8 shows mitochondrial “swapping” alters apoptosis susceptibility. Microinjection of approximately 5×10³ FVB mitochondria (Mito) into B6C3F1 oocytes increases apoptosis over that observed in non-injected (Control) or vehicle-injected B6C3F1 oocytes during a subsequent 24 h culture (A), whereas microinjection of approximately 5×10³ B6C3F1 mitochondria into FVB oocytes decreases apoptosis over that observed in non-injected (Control) or vehicle-injected FVB oocytes during a subsequent 24 h culture (B). Values are the mean ±SEM of combined data from analysis of the total number of oocytes indicated per group over each bar (asterisk, P <0.05 versus Control or Vehicle).

FIG. 9 shows electron microscopy-based tomographic reconstructions of B6C3F1 oocyte mitochondria. (A) A 1.1 nm slice from a 3-dimensional reconstruction of a volume of a B6C3F1 oocyte with a cluster of mitochondria visible. The mitochondrial matrices are dark, indicating an absence of matrix swelling. The mitochondria typically display a dichotomy of cristae structure. One-half of the dichotomy is a single, large, vacuolated cristal compartment (asterisks), whereas the other half consists of one to four lamellar compartments. One of these lamellar cristae is always observed around the periphery of the mitochondrion (arrowheads). Contacts between mitochondria and the endoplasmic reticulum are often observed (arrows). Scale bar represents 500 nm. (B) Smaller cube through the volume showing three perpendicular faces through the interior of two of the mitochondria shown in 9A. The lamellar cristae are often observed as arches or shells that extend around a large portion of the periphery (arrowheads). The vacuolated crista connects to a peripheral crista via a more tubular junction (arrow). (C) Smaller cube through the volume showing three perpendicular faces through the interior of another mitochondria shown in 9A that was volume segmented. All three faces show examples of crista junctions. Two of the crista junctions connect the peripheral crista to the intermembrane space (arrowheads). The other crista junction connects the vacuolated crista to the same space on the other side of the mitochondrion (arrow). (D) A 1.1 nm slice through the mitochondrion shown in 9C. It emphasizes a typical example of many crista junctions connecting one broad side of the peripheral crista to the intermembrane space. There are six crista junctions seen in this thin slice (arrowheads). Scale bar represents 100 nm. (E) Another thin slice through the same reconstruction shown in 6D. Note how the large vacuolated crista connects via a tubular opening (arrowhead) to the peripheral crista, making them essentially one crista, albeit with compartments of different architecture. Because of the interconnectedness, this mitochondrion possessed only the one crista. Scale is the same as that shown in 9D. (F) The mitochondrion in (9C-E) was segmented along its membranes. Even though contiguous, the large, vacuolated cristal compartment (white) was segmented separately from the lamellar cristal compartment (yellow) to aid in the analysis. The outer membrane is shown in blue and made translucent to better visualize the crista. (G, H) Two views looking from the inside and outside, respectively, of the matrix portion, between the intermembrane space and the peripheral cristal compartment. The matrix is curved and has a fenestrated appearance because of the crista junctions. There are eleven crista junctions; for reference, the arrowhead in each panel indicates the same junction. (I) A single view of the peripheral crista on the left and the vacuolated crista in the center and right. These structures are joined by a tubular connection at only one end (arrow). The vacuolated portion of the crista has fewer crista junctions, although about the same surface area as the lamellar portion of the crista. Two of the few crista junctions for the vacuolated crista are indicated (arrowheads).

FIG. 10 shows electron microscopy-based tomographic reconstructions of FVB oocyte mitochondria. (A) A 1.1 nm slice from a 3-dimensional reconstruction of a volume of a FVB oocyte showing mitochondria with a spectrum of ultrastructural anomalies. The two mitochondria labeled “I” are the types most commonly seen. The vacuolated cristal compartment prevalent in B6C3F1 mitochondria (FIG. 9) is noticeably missing. The matrix occupies the central volume; however, the cristae appear to have degraded to “onion-like” whorls seen around the periphery. The mitochondrion labeled “2” has a much-reduced matrix and in its place whorls of membranes are found. Its periphery also has whorls similar to those of the mitochondria labeled “1”. However, this mitochondrion does have a vacuolated crista (arrowhead) that nevertheless is abnormal in that it possesses internal “blobs” that may be orphaned matrix sub-volumes. The mitochondrion labeled “3” appears more like that in B6C3F1 oocytes, with the exception that it too lacks the large vacuolated cristal compartment. There is evidence of limited, localized “whorling” membrane degradation; however, most of the lamellar cristal compartment appears nearly normal. Arrows note contacts between mitochondria and vesicles, which may be derived from the endoplasmic reticulum. Scale bar represents 500 nm. (B) A 1.1 nm slice through the lower volume of mitochondrion “I” shown in FIG. 10A. The central matrix compartment (asterisk) is surrounded by whorls of membranes likely from degraded cristae. The outer membrane is ruptured (arrowheads) allowing the inner boundary membrane to extend outward (arrow). Scale bar represents 100 nm. (C) The segmented and surface-rendered mitochondrion shown in 10B. The outer membrane is shown in blue, the inner boundary membrane in grey/white, and the membrane whorls in yellow. This segmented volume shows that the whorls extend throughout the volume and are almost concentric in nature. (D, E) Two side views of the surface-rendered volume from FIG. 10C that emphasize the extent of outer membrane tearing (arrowhead in D) and blowout of the inner boundary membrane (arrowhead in E). (F, G) Two perpendicular views (top and side) of the subset of membrane whorls in I OC that are broken. (H) A 1.1 nm slice from a 3-dimensional reconstruction of a volume of a pyruvate-treated FVB oocyte showing mitochondria with similar architecture to those in B6C3F1 oocytes (FIG. 9). The large vacuolated cristal compartment (asterisk) and transverse and peripheral cristae (arrowheads) are present in the same dichotomy. Scale bar represents 500 nm. (I) The segmented and surface-rendered mitochondrion shown at the bottom in 10H. The outer membrane is shown in blue and made translucent to better visualize the cristae, the vacuolated crista in yellow, and the two lamellar cristae (arrowheads) in magenta and cyan. The transverse cristae extend less than halfway through the depth of the mitochondrion. (J) The large vacuolated crista of the mitochondrion in FIG. 10H as seen from the other side. This view emphasizes the four crista junctions (arrowheads) that connect this crista to the intermembrane space. (K) Pyruvate reverses the occurrence of abnormal mitochondrial ultrastructures in FVB oocytes. Values are the mean ±SEM of combined transmission EM data from analyzing 10-12 random sections of oocytes in each group (the total number of oocytes analyzed is indicated over each bar; different letters, P <0.05).

FIG. 11 shows cytochrome c synergizes with Smac/DIABLO to promote germ cell apoptosis. (A) Effect of microinjecting vehicle, cytochrome c (Cyt-c), recombinant Smac/DIABLO (Smac) or cytochrome c and Smac/DIABLO on the incidence of apoptosis in B6C3F1 oocytes following a 24 h culture. Values are the mean ±SEM of combined data from analyzing the total number of oocytes indicated over each bar (different letters, P <0.05). (B) Incidence of apoptosis in oocytes collected from F5 generation FVB mice expressing (Smac/DIABLO wild-type or Smac WT; n=14 mice) or lacking (Smac/DIABLO deficient or Smac KO; Smac KO; n=13 mice) functional Smac/DIABLO. Values are the mean ±SEM of combined data from analyzing the total number of oocytes indicated over each bar (asterisk, P <0.05 by chi-square analysis).

FIG. 12 shows the effect of genetic strain on general mitochondrial metabolic parameters. (A) Mitochondrial membrane potential (MMP) in oocytes of the indicated genetic backgrounds. Values are the mean I SEM of combined data from analyzing the total number of oocytes indicated per group over each bar (asterisks, P <0.05 versus B6C3F1). (B) Bioreduction potential of oocytes (MTT assay using replicate pools of 25 oocytes; the total number of oocytes analyzed per group is provided over each bar, representing the mean ±SEM of the combined data) collected from female B6C3F1, FVB or AKR/J mice (asterisk, P <0.05 versus B6C3F1 or FVB). (C) Levels of ATP in replicate pools of 25 oocytes collected from female B6C3F1, FVB or AKR/J mice (the total number of oocytes analyzed per group is provided over each bar). (D) Reduced glutathione (GSH) content in B6C3F1 (white bars), FVB (black bars) or AKR/J (gray bars) oocytes prior to (baseline) and after exposure to hydrogen peroxide (oxidative insult), without or with a brief recovery period. Values are the mean ±SEM of combined data from analyzing the total number of oocytes indicated per group over each bar.

FIG. 13 shows microinjection of embryonic stem cell mitochondria suppresses FVB oocyte death. (A) Levels of reactive oxygen species (ROS) in FVB oocytes microinjected with vehicle or approximately 1×10³ mitochondria (Mito) collected from mouse embryonic stem cells (ESC). Values are the mean ±SEM of combined data from analyzing the total number of oocytes indicated per group over each bar (asterisk, P <0.05 versus Vehicle). (B) Incidence of apoptosis in cultures of non-injected FVB oocytes (Control) or FVB oocytes microinjected with vehicle or approximately 1×10³ ESC mitochondria. Values are the mean ±SEM of combined data from analyzing the total number of oocytes indicated per group over each bar (asterisk, P <0.05 versus Control or Vehicle).

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); and B. Perbal, A Practical Guide to Molecular Cloning (1984).

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “a Bcl-2 family protein” includes a mixture of two or more Bcl-2 family proteins.

A “mitochondrial-associated protein” refers to a protein associated with mitochondrial ultrastructure that influences apoptosis and/or a protein associated with mitochondria that is involved in the regulation of mammalian preimplantation embryo survival. In aspects, the term refers to proteins associated with the apoptotic process including without limitation Bcl-2 family proteins or Bcl-2 protein interacting partners.

A “Bcl-2 family protein” refers to a member of a family of proteins that control (repress or activate) biochemical pathways of the apoptotic process. [See Adams, 2001; Green, 1998; and Vaux, 1999]. The family includes pro-survival, death-inhibitory or cell death suppressor members (Bcl-2, Bcl-xL, Bcl-w, Ced-9, Mcl-1, Aven and Diva) as well as cell death inducers (Bax, Bak, Bok/Mtd, Bcl-xS, Bad, Bim, Bik, Bid, Hrk, Noxa) (See Adams, 2001; Adams, 1998; Kroemer G. Nat. Med. 3:614-620, 1997; Reed J C. Nature 387:773-776, 1997.) Members of the family are powerful regulators of cell death. For example, Bcl-2 can protect cells from a wide array of insults, and can inhibit both apoptotic and necrotic modes of cell death (Shimizu S. Nature 374:811-813, 1995; Ziv 1, et al. Apoptosis 2:149-155, 1997). The amino acid sequence of a Bcl-2 family member has characteristic regions (See also Yin X M, et al. Nature 369:321-323, 1994; Sedlak T W, et al., Proc. Natl. Acad. Sci. USA, 92:7834-7838, 1995; Cheng E H, et al., Nature 379:554-556, 1996; Chittenden, T et al., EMBO J. 14:5589-5596, 1995; Hunter J. at al. J. Biol. Chem. 271:8521-8524, 1996; Wang K, et al., Genes Dev. 10:2859-1869, 1996). The following are some characteristic regions:

1. A hydrophobic C-terminal region, which allows for membrane anchoring.

2. BH1 and BH2: Domains which are important for formation of a hydrophobic binding cleft, where protein-protein interactions take place.

3. BH3: The C-terminal half of the amphipathic Bcl-xL second helix is part of the hydrophobic binding cleft. The homologous region in the death-inducing family members, is a ligand region, and is important for protein—protein interactions with other proteins within the Bcl-2 family.

4. A PEST-like region in Bcl-2 and Bcl-xL that is flexible, cytosol-exposed and serves as a regulator region. The region includes serine phosphorylation sites.

5. BH4: An N-terminal region, that stabilizes the three dimensional protein structure, as well as a critical docking region for several proteins including Raf-1, Bag-1 and Ced-4.

A Bcl-2 family protein includes native sequence or isolated or substantially pure polypeptides, oligopeptides, peptides, isoforms, analogues, derivatives, chimeric polypeptides, fragments, and variants thereof, or pharmaceutically acceptable salts thereof. The term particularly refers to the amino acid sequences obtained from humans, from any source whether natural, synthetic, semi-synthetic, or recombinant.

In particular aspects of the invention, a Bcl-2 family protein is a pro-survival Bcl-2 family protein, more particularly a recombinant Bcl-2 family protein, most particularly a recombinant pro-survival Bcl-2 family protein. In embodiments, the Bcl-2 family member comprises or is selected from the group consisting of BCl-2, Bcl-xL, Bcl-w, Mcl-1, A1, and Aven, and Diva or a derivative or analogue thereof. In particular embodiments of the invention, the Bcl-2 family member is Bcl-xL or Mcl-1 or a derivative or analogue thereof, in particular Bcl-xΔC that lacks the C terminus or Bcl-xES lacking the BH3/BH1 region. In other particular embodiments, the Bcl-2 family member is a phosphorylation or caspase cleavage mutant [Grethe, 2004; Domina, 2000; Domina, 2004, Clohessy, 2004; Michels, 2004].

Amino acid sequences for Bcl-2 family proteins (and nucleic acids encoding the proteins) are available in public databases such as the NCBI and SwissProt databases. Accession numbers for amino acid and nucleic acid sequences for exemplary pro-survival Bcl-2 family proteins are listed in Table 1.

A “Bcl-2 protein interacting partner” refers to a substance (e.g. protein) that directly or indirectly interacts with a Bcl-2 family protein, especially a pro-survival Bcl-2 family protein, (e.g. Bcl-2) to protect against apoptosis. An example of a Bcl-2 protein interacting partner includes without limitation Bag-1 (NCBI Gene ID. No. 573, Accession Nos. AAC34258, NP_{—}004314, CAH72516-CAH72520, CAH72741-CAH72743, AAC34258, AAD25045, BAD96469, AAH01936, AAH14774, or AAD11467).

A “genomic integrity modifier protein” refers to a protein that is involved in maintaining cellular integrity of cells to reduce or prevent cellular transformation or death. A genomic integrity modifier protein is especially a DNA repair protein, i.e. a protein involved in repair of double strand DNA breaks via homologous recombination or non-homologous end joining [Friedburg, E et al., DNA Repair and Mutagenesis, ASM Press, Washington D.C., 1995; Nickollof J and Hoekstra, M., DNA Damage and Repair, Humana Press, Totowa, N.J., 1998]. In aspects of the invention, a genomic integrity modifier protein is a protein involved in repair of double strand DNA breaks through homologous recombination. In particular aspects of the invention, the genomic integrity modifier protein is a RecA family protein.

A “RecA family protein” is a member of a family of proteins that share a structural motif known as the “RecA signature sequence” or “Domain II” which forms the ATP binding sites. Examples of RecA family proteins are disclosed in Sandler, S J, et al., Nucl Acids Res 24:2125-2132 (1996); Roca, A 1, et al., Crit Rev Biochem Mol Biol 25:415-456 (1990); Eisen, J A, J. Mol. Evol. 41:1105-1123 (1995); Lloyd, A T, et al., J. Mol. Evol. 37:399-407 (1993) Seitz, E M, et al., Genes Dev. 12:1248-1253 (1998); and Bianco, P R, et al., Frontiers Biosci. 3:570-603 (1998).

In aspects of the invention, a RecA family protein is a Rad51 family protein. A “rad51 family protein” is a RecA family protein comprising an N-terminal extension (Ogawa et al, Cold Spring Harbor Symp. On Quant. Biol., Vol. LVIII pp. 567-576, 1993; Johnson R D & Symington, L S, Mol. Cell. Biol 15:4843-4850, 1995). In particular aspects of the invention the Rad51 family protein is a recombinant protein. A Rad51 family protein includes without limitation DMC1, LIM15, Rad55, Rad57, Rad50, Rad52, Rad54, Rad55, Rad59, MRE11, and XRS2, especially Rad51. Rad 51 is a 339 amino acid protein (36966 Da) which is localized in the nuclear compartment and colocalizes with RAD51API to multiple nuclear foci upon induction of DNA damage (Benson, 1994). Rad51 interacts with many different proteins including: BRCA1, BRCA2, p53, XRCC3, RAD54L, RAD54B, RAD51API, and CHEK1/CHK1.

A genomic integrity modifier protein, in particular a Rad51 family protein, includes native sequence or isolated or substantially pure polypeptides, oligopeptides, peptides, isoforms, analogues, derivatives, chimeric polypeptides, fragments, and variants thereof, or pharmaceutically acceptable salts thereof. The term particularly refers to the amino acid sequences obtained from humans, from any source whether natural, synthetic, semi-synthetic, or recombinant.

Amino acid sequences for RecA family proteins, in particular Rad51 family proteins, (and nucleic acids encoding the proteins) are available in public databases such as the NCBI and SwissProt databases. Accession numbers for amino acid and nucleic acid sequences for exemplary Rad51 family proteins are listed in Table 2.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The terms “substantially pure” or “isolated,” refer to mitochondrial-associated proteins or genomic integrity modifier proteins that are separated as desired from RNA, DNA, proteins or other contaminants with which they are naturally associated. For example, when referring to proteins and polypeptides, a protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least about 75%, at least about 80%, at least about 85%, more preferably, at least about 90%, at least about 95% of the total protein. Preferably, the protein will make up greater than about 90%, and more preferably, greater than about 95% of the total protein in the composition.

An “isoform” refers to a polypeptide that contains the same number and kinds of amino acids as a mitochrondrial-associated protein and/or genomic integrity modifier protein, but the isoform has a different molecular structure. Isoforms preferably have the same properties (e.g., biological and/or immunological activity) as a mitochrondrial-associated protein and/or genomic integrity modifier protein.

An “analogue” includes a polypeptide wherein one or more amino acid residues of a native polypeptide have been substituted by another amino acid residue, one or more amino acid residues of a native polypeptide have been inverted, one or more amino acid residues of the native polypeptide have been deleted, and/or one or more amino acid residues have been added to the native polypeptide. Such an addition, substitution, deletion, and/or inversion may be at either of the N-terminal or C-terminal end or within the native polypeptide, or a combination thereof.

A “derivative” includes a polypeptide in which one or more of the amino acid residues of a native polypeptide have been chemically modified. A chemical modification includes adding chemical moieties, creating new bonds, and removing chemical moieties. In particular, a chemical modification can include internal linkers (e.g. spacing or structure-inducing) or appended molecules, such as molecular weight enhancing molecules (e.g., polyethylene glycol, polyamino acid moieties, etc.,), or tissue targeting molecules. A polypeptide may be chemically modified, for example, by alkylation, acylation, glycosylation, pegylation, ester formation, deamidation, or amide formation.

Native-sequence polypeptides may be modified to make analogues or derivatives that are more active or have longer half lives (e.g. by making them resistant to degradation or to reduce metabolic clearance).

A “variant” refers to a polypeptide having at least about 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Such variants include for instance polypeptides wherein one or more amino acid residues are added to, or deleted from the N- or C-terminus of the full-length or mature sequences of the polypeptide, including variants from other species. A naturally occurring allelic variant may contain conservative amino acid substitutions from the native polypeptide sequence or it may contain a substitution of an amino acid from a corresponding position in a polypeptide homolog, for example, a murine polypeptide. “Identity” as known in the art and used herein, is a relationship between two or more amino acid sequences as determined by comparing the sequences. It also refers to the degree of sequence relatedness between amino acid sequences as determined by the match between strings of such sequences. Identity and similarity are well known terms to skilled artisans and they can be calculated by conventional methods (for example, see Computational Molecular Biology, Lesk, A. M. ed., Oxford University Press, New York, 1988; Biocomputing: Informatics and Genome Projects, Smith, D. W. ed., Academic Press, New York, 1993; Computer Analysis of Sequence Data, Part 1, Griffin, A. M. and Griffin, H. G. eds., Humana Press, New Jersey, 1994; Sequence Analysis in Molecular Biology, von Heinje, G. Academic Press, 1987; and Sequence Analysis Primer, Gribskov, M. and Devereux, J. eds. M. Stockton Press, New York, 1991, Carillo, H. and Lipman, D., SIAM J. Applied Math. 48:1073, 1988). Methods which are designed to give the largest match between the sequences are generally preferred. Methods to determine identity and similarity are codified in publicly available computer programs including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990).

Mutations may be introduced into a polypeptide by standard methods, such as site-directed mutagenesis and PCR-mediated mutagenesis. Conservative substitutions can be made at one or more predicted non-essential amino acid residues. A conservative amino acid substitution is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Computer programs, for example DNASTAR, may be used to determine which amino acid residues may be substituted, inserted, or deleted without abolishing biological and/or immunological activity.

A “fragment” or “portion” of a polypeptide may range in size from four amino acids to the entire amino acid minus one amino acid. A fragment or portion of a polypeptide can be a polypeptide which is for example, about 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide. A fragment can be a domain of a polypeptide.

In the context of the present invention a mitochrondrial-associated protein and/or genomic integrity modifier protein also includes an agonist of the protein. “Agonist” refers to an agent that mimics (i.e., mimetics) or upregulates (e.g. potentiates or supplements) a Bcl-2 family protein activity and/or genomic integrity modifier protein activity, in particular a biological and/or immunological activity of a Bcl-2 family protein and/or genomic integrity modifier protein. An agonist can be a native mitochrondrial-associated protein and/or genomic integrity modifier protein or derivative thereof having at least one biological activity of a native protein. An agonist can be a compound that upregulates expression of a protein or which increases at least an activity of a protein. An agonist can also be a compound that increases the interaction of a protein and another molecule. Agonists include molecules that bind to a mitochrondrial-associated protein and/or genomic integrity modifier protein.

“Mimetic” refers to a synthetic chemical compound that has substantially the same structural and/or functional characteristics of a mitochrondrial-associated protein and/or genomic integrity modifier protein. A mimetic can be composed entirely of synthetic, non-natural analogues of amino acids, or, is a chimeric polypeptide of partly natural peptide amino acids and partly non-natural analogues of amino acids. A polypeptide can be characterized as a mimetic when all or some of its residues are joined by chemical means other than natural peptide bonds (see, e.g., Spatola (1983) in Chemistry and Biochemistry of Amino Acids, Peptides and Proteins, Vol.7, pp 267-357, “Peptide Backbone Modifications,” Marcell Dekker, N.Y.). Mimetics also include peptoids, oligopeptoids (Simon et al (1972) Proc. Natl. Acad, Sci USA 89:9367); and peptide libraries containing peptides of a designed length representing all possible sequences of amino acids corresponding to a motif or peptide. A particular mimetic refers to a molecule, the structure of which is developed based on the structure of a mitochrondrial-associated protein and/or genomic integrity modifier protein or portions thereof, and is able to effect some of the actions of chemically or structurally related molecules.

A “chimeric polypeptide” comprises all or part (preferably biologically active) of a mitochrondrial-associated protein and/or genomic integrity modifier protein operably linked to a heterologous polypeptide (i.e., a polypeptide other than the same mitochrondrial-associated protein and/or genomic integrity modifier protein). Within the chimeric polypeptide, the term “operably linked” is intended to indicate that the mitochrondrial-associated protein and/or genomic integrity modifier protein and the heterologous polypeptide are fused in-frame to each other. The heterologous polypeptide can be fused to the N-terminus or C-terminus of the mitochrondrial-associated protein and/or genomic integrity modifier protein. A useful chimeric polypeptide is a GST fusion protein in which a mitochrondrial-associated protein and/or genomic integrity modifier protein is fused to the C-terminus of GST sequences. Another example of a chimeric polypeptide is an immunoglobulin fusion protein in which all or part of a mitochrondrial-associated protein and/or genomic integrity modifier protein is fused to sequences derived from a member of the immunoglobulin protein family. Chimeric polypeptides can be produced by standard recombinant DNA techniques.

A mitochrondrial-associated protein and/or genomic integrity modifier protein can be prepared by a variety of methods known in the art such as solid-phase synthesis, purification of the proteins from natural sources, recombinant technology, or a combination of these methods. See for example, U.S. Pat. Nos. 5,188,666, 5,120,712, 5,523,549, 5,512,549, 5,977,071, 6,191,102, Dugas and Penney 1981, Merrifield, 1962, Stewart and Young 1969, and the references cited herein. Derivatives can be produced by appropriate derivatization of an appropriate backbone produced, for example, by recombinant DNA technology or peptide synthesis (e.g. Merrifield-type solid phase synthesis) using methods known in the art of peptide synthesis and peptide chemistry. In aspects of the invention a mitochondrial-associated protein and/or genomic integrity modifier protein is an isolated or purified protein, a recombinant protein, or a synthesized protein. “Modulate” or “modulating” refers to a change or an alteration in genetic integrity and/or mitochondrial ultrastructure that influences apoptosis. In aspects, the term refers to a change or an alteration in the activity of a mitochrondrial-associated protein and/or genomic integrity modifier protein, in particular the biological activity of a Bcl-2 family protein and/or a Rad51 family protein. Modulation may be an increase or decrease in the activity of a protein, a change in binding characteristics, or any other changes in the biological, functional, or immunological properties of a protein. In some aspects, the terms refer to enhancing developmental potential of oocytes. In other aspects, the terms refer to decreasing, inhibiting or reversing reduced DNA repair capacity, DNA damage, mitochondrial defects, and/or deficiencies in ROS production in oocytes or preimplantation embryos.

“Biological activity” refers to structural, regulatory, or biochemical functions of a naturally occurring molecule.

The term “oocytes” refers to the gamete from the follicle of a female animal, whether vertebrate or invertebrate. The animal is preferably a mammal, including a human, non-human primate, a bovine, equine, porcine, ovine, caprine, buffalo, guinea pig, hamster, rabbit, mice, rat, dog, cat, or a human. Suitable oocytes for use in the invention include immature oocytes, and mature oocytes from ovaries stimulated by administering to the oocyte donor, in vitro or in vivo, a fertility agent(s) or fertility enhancing agent(s) (e.g. inhibin, inhibin and activin, clomiphene citrate, human menopausal gonadotropins including FSH, or a mixture of FSH and LH, and/or human chorionic gonadotropins). In some embodiments of the invention, the oocytes are aged (e.g. from humans 40 years +, or from animals past their reproductive prime). The oocytes in some embodiments of the invention contain mitochondrial DNA mutations or mutations in genes involved in DNA repair. Methods for isolating oocytes are known in the art.

In the nuclear transfer embodiments of the invention oocytes are used as recipient cells (such cells are referred to herein as “recipient oocytes”). The recipient ooctyes are obtained from mammals, especially non-human mammals, in particular domestic, sports, zoo, and pet animals including but not limited to bovine, ovine, porcine, equine, caprine, buffalo, and guinea pigs, rabbits, mice, hamsters, rats, primates, etc.

“Preimplantation embryo” refers to the very early free-floating embryo of an animal, from the time the oocyte is fertilized (zygote), until the beginning of implantation (in humans, a period of about 6 days). The term also includes embryos resulting from nuclear transfer, in all the development stages through the blastocyst stage. A preimplantation embryo may be from a vertebrate or an invertebrate, preferably a mammal, more preferably a human, a non-human primate, a bovine, equine, porcine, ovine, caprine, buffalo, guinea pig, hamster, rabbit, mice, rat, dog, or cat.

The term “zygote” refers to a fertilized oocyte prior to the first cleavage division.

The expression “enhancing the developmental potential of oocytes” refers to increasing the quality of the oocyte so that it will be more capable of being fertilized and/or enhancing mitochondrial function or activity in the oocyte for subsequent development and reproduction. Increasing the quality of the oocyte, and thus the fertilized oocyte (e.g. zygote), preferably results in enhanced development of the oocyte into an embryo and its ability to be implanted and form a healthy pregnancy. The expression “enhancing the developmental potential of preimplantation embryos” refers to increasing the quality of the preimplantation embryos and/or enhancing mitochondrial function or activity in the preimplantation embryos for subsequent development and reproduction. Increasing the quality of the preimplantation embryos, preferably results in enhanced development of the preimplantation embryos into an embryo and their ability to be implanted and form a healthy pregnancy. Quality can be assessed by the appearance of the developing embryo by visual means and by the IVF or nuclear transfer success rate. Criteria to judge quality of the developing embryo by visual means include, for example, their shape, rate of cell division, fragmentation, appearance of cytoplasm, and other means recognized in the art of IVF and nuclear transfer.

“Spermatozoa” refers to male gametes that can be used to fertilize oocytes.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. include but are not limited to saline, buffered saline, dextrose, water, glycerol, ethanol, and combinations thereof. The use of such media and agents for an active substance is well known in the art.

### DESCRIPTION OF EMBODIMENTS OF THE INVENTION

The present invention generally involves the use of mitochondrial-associated proteins and/or genomic integrity modifier proteins to enhance the developmental potential of animal oocytes and preimplantation embryos, especially mammals, including sports, zoo, pet, and farm animals, in particular dogs, cats, cattle, pigs, horses, goats, buffalo, rodents (e.g. mice, rats, guinea pigs), monkeys, sheep, and humans, especially humans. In the nuclear transfer methods, mitrochondrial-associated proteins and/or genomic integrity modifier proteins are used to enhance the developmental potential of recipient oocytes, especially non-human recipient oocytes.

Methods of the invention involve removing the oocytes from follicles in the ovary. This can be accomplished by conventional methods for example, using the natural cycle, during surgical intervention such as oophorohysterectomy, during hyperstimulation protocols in an IVF program, or by necropsy. Oocyte removal and recovery can be suitably performed using transvaginal ultrasonically guided follicular aspiration.

In a method of the invention for enhancing developmental potential of oocytes, mitochondrial-associated proteins and/or genomic integrity modifier proteins are introduced into the oocytes, or the oocytes can be cryopreserved for storage in a gamete or cell bank. If the oocytes are not cryopreserved the oocytes can be treated in accordance with the method of the invention preferably within 48 hours after aspiration. If the oocytes are frozen, they can be thawed when it is desired to use them and treated in accordance with a method of the invention.

Mitochondrial-associated proteins and/or genomic integrity modifier proteins may be introduced into the oocytes (or zygotes) by conventional microinjection techniques, electroporation, methods using viral fusion proteins or cationic lipids, and methods devised by a person skilled in the art (see for example, Protein Delivery: Physical Systems, Sanders and Hendren (eds) (Plenum Press, 1997). The proteins may be introduced into the cytoplasm, the pronucleus of an oocyte, or the pronucleus of a zygote (in particular the male pronucleus).

Mitochondrial-associated proteins and/or genomic integrity modifier proteins may be formulated as pharmaceutical compositions which can be prepared by per se known methods for the preparation of pharmaceutically acceptable compositions. Suitable pharmaceutically acceptable carriers, excipients and vehicles are described, for example, in Remington's Pharmaceutical Sciences, 19^{th} Edition (Mack Publishing Company, Easton, Pa., USA 1995). On this basis, the compositions include, albeit not exclusively, solutions of the proteins in association with one or more pharmaceutically acceptable vehicles or diluents, and contained in buffered solutions with a suitable pH and iso-osmotic with the physiological fluids. A mitochondrial-associated protein and/or genomic integrity modifier protein may be formulated in a pharmaceutically acceptable delivery composition that can be used in the form or a solid, a solution, an emulsion, a dispersion, a micelle, a liposome, and the like, in admixture with an organic or inorganic carrier or excipient suitable for administration to oocytes or preimplantation embryos. The mitochondrial-associated protein and/or genomic integrity modifier protein may be a concentrate including lyophilized compositions which may be diluted prior to use.

The mitochondrial-associated proteins and/or genomic integrity modifier proteins may be in the form of a kit, in particular a kit or article-of manufacture including mitochondrial-associated proteins and/or genomic integrity modifier proteins either as concentrates (including lyophilized compositions), which may be further diluted prior to use or at the concentration of use, where the vials may include one or more dosages.

In an aspect, the invention provides an article-of-manufacture comprising packaging material and a pharmaceutical composition identified for improving embryo development after in vitro fertilization or embryo transfer contained within the packaging material, the pharmaceutical composition including as an active ingredient, a mitochondrial-associated protein, in particular a Bcl-2 family protein, more particularly a pro-survival Bcl-2 family protein, and/or a genomic integrity modifier protein, in particular a RecA family protein, more particularly a Rad51 family protein, and a pharmaceutically acceptable carrier, excipient, or vehicle.

After introduction, simultaneously with, or prior to the introduction of the mitochondrial-associated proteins and/or genomic integrity modifier proteins, the oocytes are fertilized with suitable spermatozoa from the same species. The fertilization can be carried out by known techniques including sperm injection, in particular intracytoplasmic sperm injection (ICSI). In ICSI, sperm is injected directly into an oocyte with a microscopic needle.

In an embodiment, the oocytes are simultaneously injected with mitochondrial-associated proteins and/or genomic integrity modifier proteins and sperm. In another embodiment, oocytes are fertilized with sperm followed by introduction of the proteins into the fertilized oocytes (zygotes).

The fertilized oocytes (zygotes) can be cultured or immediately transferred to the subject. Suitable human in vitro fertilization and embryo transfer procedures that can be used include in vitro fertilization (IVF) (Trounson et al. Med J Aust. 1993 Jun 21; 158(12):853-7, Trouson and Leeton, in Edwards and Purdy, eds., Human Conception in Vitro, New York:Academic Press, 1982, Trounson, in Crosignani and Rubin eds., In Vitro Fertilization and Embryo Transfer, p. 315, New York: Academic Press, 1983); intracytoplasmic sperm injection (ICSI) (Casper et al., Fertil Steril. 1996 May;65(5):972-6); in vitro fertilization and embryo transfer (IVF-ET)(Quigly et al, Fert. Steril., 38: 678, 1982); gamete intrafallopian transfer (GIFT) (Molloy et al, Fertil. Steril. 47: 289, 1987); and pronuclear stage tubal transfer (PROST) (Yovich et al., Fertil. Steril. 45: 851, 1987). Generally, in IVF methods the fertilized oocytes are introduced into the uterus of the subject while in other methods such as GIFT and ZIFT the fertilized oocytes are transferred to a fallopian tube.

The methods and compositions of the invention can be used to increase the success rate of embryo development. While not wishing to be bound by a particular theory, the outcomes of introducing mitochondrial-associated proteins and/or genomic integrity modifier proteins will increase the concentration of cell death suppressors, improve mitochondrial physiology and metabolism, decrease, reverse or inhibit the reduction of DNA repair capacity, DNA damage and/or deficiencies in ROS production, providing embryos with protection from apoptosis or arrest during the critical early embryonic stages of development. Recombinant mitochondrial-associated proteins and/or genomic integrity modifier proteins may have a particular advantage in that they have a terminal half-life resulting in their clearance prior to implantation. Thus, they provide transient support during the time most susceptible to embryo demise and they should not result in any genetic modification of offspring. In addition, recombinant mitochondrial-associated proteins and/or genomic integrity modifier proteins are stable, can be lyophilized and reconstituted at the time of injection, and can be combined with mechanical sperm injection, resulting in a clinically feasible, widely available treatment requiring no extra equipment or skills apart from those needed for intracytoplasmic sperm injection (ICSI).

The invention also contemplates improved nuclear transfer methods using mitochondrial-associated proteins and/or genomic integrity modifier proteins. Nuclear transfer methods or nuclear transplantation methods are known in the literature and are described in for example, Campbell et al, Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci., USA, 90:6143-6147 (1993); WO 94/26884; WO 94/24274, WO 90/03432, U.S. Pat. Nos. 4,944,384 and 5,057,420.

Methods for isolation of recipient oocytes suitable for nuclear transfer methods are well known in the art. Generally, the recipient oocytes are surgically removed from the ovaries or reproductive tract of a mammal, e.g., a bovine. Once the oocytes are isolated they are rinsed and stored in a preparation medium well known to those skilled in the art, for example buffered salt solutions

Recipient oocytes must generally be matured in vitro before they may be used as recipient cells for nuclear transfer. This process generally requires collecting immature (prophase I) oocytes from mammalian ovaries, and maturing the oocytes in a maturation medium prior to fertilization or enucleation until the oocyte attains the metaphase II stage. Metaphase II stage oocytes, which have been matured in vivo, may also be used in nuclear transfer techniques.

Enucleation of the recipient oocytes may be carried out by known methods, such as described in U.S. Pat. No. 4,994,384. For example, metaphase II oocytes may be placed in HECM, optionally containing cytochalasin B, for immediate enucleation, or they may be placed in a suitable medium, (e.g. an embryo culture medium), and then enucleated later, preferably not more than 24 hours later. Enucleation may be achieved microsurgically using a micropipette to remove the polar body and the adjacent cytoplasm (McGrath and Solter, Science, 220:1300, 1983), or using functional enucleation (see U.S. Pat. No. 5,952,222). The recipient oocytes may be screened to identify those which have been successfully enucleated.

The recipient oocytes may be activated on, or after nuclear transfer using methods known to a person skilled in the art. Suitable methods include culturing at sub-physiological temperatures, applying known activation agents (e.g. penetration by sperm, electrical and chemical shock), increasing levels of divalent cations, or reducing phosphorylation of cellular proteins (see U.S. Pat. No. 5,496,720).

A nucleus of a donor cell, preferably of the same species as the enucleated oocyte, is introduced into the enucleated recipient oocyte. The donor cell nucleus may be obtained from any mammalian cells. Donor cells may be differentiated mammalian cells derived from mesoderm, endoderm, or ectoderm. In particular, the donor cell nucleus may be obtained from epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells. Suitable mammalian cells may be obtained from any cell or organ of the body. The mammalian cells may be obtained from different organs including skin, lung, pancreas, liver, stomach, intestine, heart, reproductive organ, bladder, kidney and urethra.

The nucleus of the donor cell is preferably membrane-bounded. A donor cell nucleus may consist of an entire blastomere or it may consist of a karyoplast. A karyoplast is an aspirated cellular subset including a nucleus and a small amount of cytoplasm bounded by a plasma membrane. (See Methods and Success of Nuclear Transplantation in Mammals, A. McLaren, Nature, Volume 109, Jun. 21, 194 for methods for preparing karyoplasts).

Mitochondrial-associated proteins and/or genomic integrity modifier proteins are introduced into the enucleated recipient oocyte. The proteins are preferably derived from the same species as the donor cell, more preferably from the same species and cell type as the donor cell, and most preferably from the same individual from which the donor cell nucleus is derived. Methods for preparing the proteins are known to a person skilled in the art.

Donor cells may be propagated, genetically modified, and selected in vitro prior to extracting the nucleus.

The nucleus of a donor cell may be introduced into an enucleated recipient oocyte using micromanipulation or micro-surgical techniques known in the art (see McGrath and Solter, supra). For example, the nucleus of a donor cell may be transferred to the enucleated recipient oocyte by depositing an aspirated blastomere or karyoplast under the zona pellucida so that its membrane abutts the plasma membrane of the recipient oocyte. This may be accomplished using a transfer pipette.

Fusion of the donor nucleus and the enucleated oocyte may be accomplished according to methods known in the art. For example, fusion may be aided or induced with viral agents, chemical agents, or electro-induced. Electrofusion involves providing a pulse of electricity sufficient to cause a transient breakdown of the plasma membrane. (See U.S. Pat. No. 4, 994,384). In some cases (e.g. with small donor nuclei) it may be preferable to inject the nucleus directly into the oocyte rather than using electroporation fusion. Such techniques are disclosed in Collas and Barnes, Mol. Reprod. Dev., 38:264-267 (1994).

The clones produced using the nuclear transfer methods as described herein may be cultured either in vivo (e.g. in sheep oviducts) or in vitro (e.g. in suitable culture medium) to the morula or blastula stage. The resulting embryos may then be transplanted into the uteri of a suitable animal at a suitable stage of estrus using methods known to those skilled in the art. A percentage of the transplants will initiate pregnancies in the surrogate animals. The offspring will be genetically identical where the donor cells are from a single embryo or a clone of the embryo.

The following non-limiting examples are illustrative of the present invention:

### Example 1

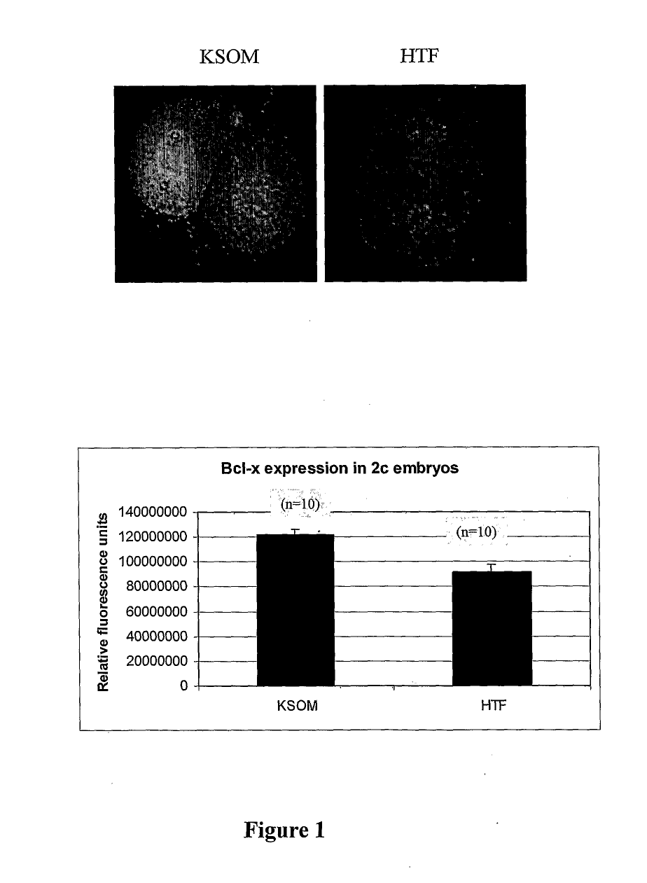

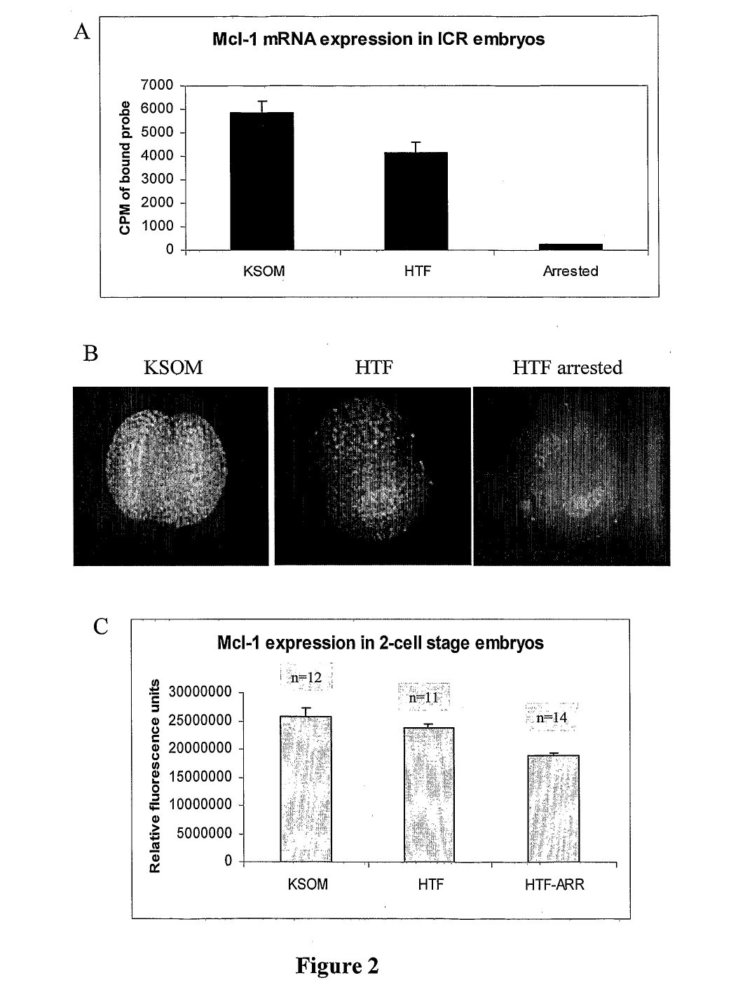

Mouse zygotes of outbred strains (such as ICR or CDI) have a limited developmental potential when cultured in media frequently used for human embryo culture (HTF, human tubal fluid). Less than half (approximately 40%) of the embryos will reach the blastocyst stage when maintained in HTF medium, with embryonic arrest observed throughout preimplantation development. While a fraction of embryos arrested at the 2-cell stage, a second major hurdle was observed around the time of compaction. In contrast, KSOM medium fully supports mouse embryo development in vitro (blastocyst formation rate >90%). The embryonic arrest at the 2-cell stage in HTF medium is accompanied by alterations in mitochondrial membrane potential and by abnormal spatial reorganization of mitochondria. Further, gene expression studies revealed that arrested embryos, maintained in HTF medium for 44 hours, fail to sustain Mcl-I expression (FIG. 1). Interestingly, Bcl-x protein, which is already expressed by the embryonic genome at the late 1-cell stage [Jurisicova, 1998], was downregulated to the same extent at the 2-cell stage prior to onset of arrest, 24 hours after HTF exposure (FIG. 2), indicating that contrary to the situation in cancer cells, Bcl-x may be upstream of Mcl-1 in preimplantation embryos. Embryonic arrest under these conditions may be partially rescued by injection of mitochondria isolated from ES cells, indicating that enrichment of the mitochondrial pool may facilitate the progression of zygotes throughout early cleavage stages.

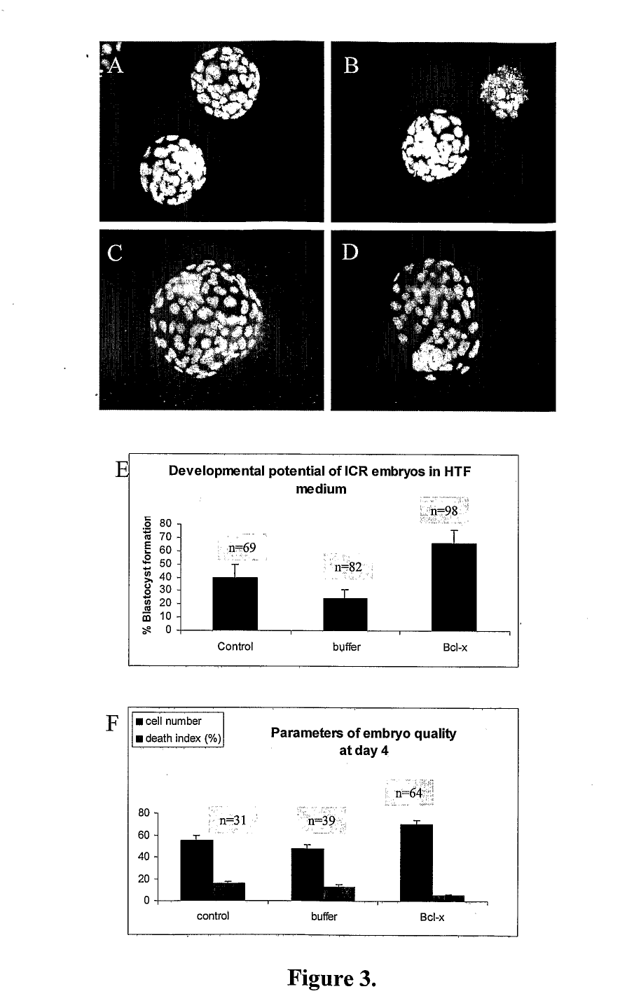

As an alternative approach, rescue of embryonic arrest in mice was attempted by microinjection of the recombinant Bcl-x protein, Bcl-xΔC, truncated at the C-terminus [Kuwana, 2002]. Results of these experiments are summarized in FIG. 3. Injection of recombinant Bcl-xΔC into one pronucleus (generally the male pronucleus) of zygotes not only overcame embryonic arrest, but resulted in blastocysts of superior quality, as reflected by a dramatic increase in cell numbers and a decrease in the rate of cell death.

### Example 2

The connection between aging and the endowment of anti- and pro-apoptotic Bcl-2 family members in human oocytes.

Proper deposition of maternal products in the oocyte is an absolute requirement for successful embryo development. Bcl-2 family members may be among the maternal signals facilitating preimplantation embryo development. A preliminary screen performed on a small set of human oocytes revealed variability in the endowment of several Bcl-2 family members (particularly Bcl-x, Mcl-1 and Bax). Moreover, Bax transcript and proteins levels have been found to be elevated in biologically aged murine oocytes [Jurisicova, 2002]. The connection between maternal age and the endowment of Bcl-2 family members will be investigated by examining oocytes from patients aged 25-40 years and correlating the expression of these genes with developmental competence in sibling embryos (i.e. arrest and fragmentation) in the ICSI/IVF cycle.

At the time of retrieval, approximately 10% of oocytes obtained from patients undergoing hormonal stimulation are immature and thus are unsuitable for fertilization with ICSI. With patient consent, these oocytes will be used immediately for this study either in GV or MI stage, without any in vitro maturation, as this may introduce further variability in the expression studies. To determine whether human oocytes with biological aging express altered levels of Bcl-2-family molecules known to be expressed by oocytes (Bcl-x, Mcl-1, Diva, Aven, Bax, Bok transcripts), real time RT-PCR will be performed. ABI prism fluorescence detecting thermocyclers will be employed. This approach requires a small amount of RNA as it can detect as little as 10 copies of transcript in the starting material, and has been used successfully to assess gene expression in human oocytes [Steuerwald, 1999]. Primer sequences and starting conditions for all studied genes will be selected using Primer Express (ABI Prism) software. Using optimized conditions, transcripts from individual human oocytes will be measured and compared. Briefly, RNA will be extracted and reversed transcribed using oligodT priming as previously described [Jurisicova, 1998]. SYBR green I, a double strand intercalating dye, will be added to the PCR mixture to detect PCR product as it accumulates during progression of the PCR cycles. Relative quantitation will be performed using the comparative relative cycle number method. SYBR green will detect both specific and non-specific accumulation of product. Thus, to verify amplification of a single product, a thermal denaturation curve of the PCR product will be generated at the end of each PCR. The shape of this curve will reveal if a single product was formed and the indicated melting temperature will provide evidence of product specificity. This will also be confirmed on select samples by agarose gel electrophoresis followed by product sequencing. Parallel reactions amplifying the housekeeping gene (18S) will serve as an internal standard to allow comparison of values across samples.

Ovulated MII oocytes frequently do not express transcriptionally available mRNA (with long poly A tails) and are, therefore, unsuitable for RT-PCR studies. Therefore, indirect immunocytochemistry using commercially available antibodies (Santa Cruz Biotechnology) will be performed on unfertilized MII oocytes (if they fail to show the signs of second polar body extrusion and formation of pronuclei), to determine changes in the protein levels for any of the transcripts showing differential accumulation with age. Samples will be analysed using deconvolution microscopy and intensity of staining will be determined using Delta Vision software. Clinical embryology data will be compared with expression patterns of all studied transcripts in order to determine whether some patients have a maternal predisposition towards abnormal embryonic development that can be attributed to altered profile of Bcl-2 family members.

It is expected that age-related decreases in pro-survival gene expression (Bcl-xL, Mcl-1, Aven and Diva) accompanied by an increase in the Bax and Bok maternal products will be observed in the human oocytes. Similar patterns may emerge for a subset of oocytes in younger patients with recurrent poor quality embryos.

### Example 3

To determine to what extent loss of maternal Bcl-x contributes to abnormal preimplantation embryo development and investigate the pathways of its action.

A. Assessment of preimplantation development in mice lacking Bcl-x.

Culture of zygotes from mice with outbred genetic backgrounds (comparable to the human population) in HTF medium leads to poor developmental performance, with only a fraction of embryos reaching the blastocyst stage. Culture in HTF medium results in a 25% decrease in the expression of Bcl-x protein compared to more favourable culture conditions in KSOM. Hence, insufficient Bcl-x expression may be responsible for unsuccessful preimplantation development. This is supported by observations that females lacking the Bcl-x gene in the ovary are subfertile as only 30% of them produce litters [Riedlinger, 2002]. Furthermore, follicular endowment, ovulation rates and luteal function in these mice are normal, suggesting that abnormal preimplantation development due to lack of maternally accumulated Bcl-x protein may be responsible for the observed phenotype.

The experiments will involve the generation of female mice with oocytes lacking Bcl-x. Mice carrying the Bcl-x gene flanked by lox P sites, Bcl-x^{f1/f1} [Rucker, 2000] will be mated with mice carrying Cre-recombinase driven by a zona pellucida-3 promotor [Lewandoski, 1997]. The resulting females (Bcl-x^{f1/de1/ZP3Cre}) will be used for mating and subsequent embryonic analysis. Cre-recombinase in the growing oocytes can excise the maternal loxP Bcl-x allele prior to fertilization and is also capable of excising the paternal allele upon fertilization [Lewandoski, 1997]. Females will be mated with either WT or Bcl-x^{f1/f1} males. Embryos will be obtained from superovulated females at the zygote stage (24 h post hCG) and will be placed in culture using more favourable culture medium (KSOM) supplemented with amino acids. Developmental competence of embryos, assessed through the rate of blastocyst formation in each group, will be recorded daily in all experiments. If the presence of abnormalities in morphology, arrest, fragmentation and development arise, the embryos will be further analyzed (see below). At day 4.5, allocation of cells to either the trophectodermal or embryonic lineage will be assessed using differential labeling combined with TUNEL analysis [Handyside, 1984]. This method allows the determination of the number of TE and ICM cells, as well as the mitotic and dead cell index [Hardy, 1989]. The correct establishment of appropriate ICM and TE cell numbers is essential to the normality of the developing conceptus [Hardy, 1997]. Deletion of bcl-x will be confirmed in embryos after cellular analysis by PCR based genotyping.

Mitochondrial endowment and function in oocytes and embryos lacking Bcl-x.

Several recent studies suggest an involvement of mitochondria in reproductive outcome associated with regulation of cell death as well as with aging (reviewed in Cummins, 2004 and Van Blerkom, 2004] To date, this work has concentrated on analysis of mitochondrial potential, energy production and mtDNA integrity/mutational load. These studies will address the issues of mitochondrial function and mtDNA copy number as an underlying mechanism of early embryo demise due to lack of maternally stored Bcl-x since this protein has been reported to dramatically affect mitochondrial function in somatic cells [Vander Heiden 1999, 2001]. Moreover, the involvement of Bcl-x in the subcellular distribution of mitochondria and its connection to mitochondrial replication will be investigated since Bcl-2 had been previously shown to inhibit replication of abnormal mitochondria [Eliseev, 2003].

Oocytes and embryos obtained from females lacking Bcl-x (stage will be chosen based on the phenotype determined in A above) will be incubated with a fluorochrome (DePsipher, R&D Systems) that allows simultaneous detection of mitochondria with low (green) and high (red) mitochondrial potential. The red/green ratio will be determined for all experimental groups of individual oocytes/embryos (n=20 per genotype), and an average ratio of J-aggregate to J-monomer staining for the entire oocyte/embryo will be determined as previously described. Mitochondrial distribution will be analyzed within 2-cell stage embryos of various genotypes and compared with wildtype embryos, since alterations in the subcellular distribution of these organelles in developmentally compromised embryos has previously been observed. Reactive oxygen species formation will be determined through the use of 2′, 7′-dichlorodihydrofluorescein diacetate (H₂DCFDA, Molecular Probes). Upon entering the cell, the acetate groups are hydrolysed, trapping a membrane impermeant form of the dye (H₂DCF). Glutathione, a thiol-containing tripeptide that acts to protect cells from free radicals, oxidants and electrophiles will be measured using the fluorescent dye Monochlorobimane (Molecular Probes). Both dyes are cell permeable chemicals used for routine quantitation of cellular ROS and glutathione content. Since the fluorescence of the dye is dependent on the amount of ROS and glutathione, samples will be analyzed using a deconvolution microscope and the amount of fluorescence will be quantitated using the Delta Vision software package (Silicon Graphics). Since oocyte mitochondria are haploid (i.e., each mitochondrion contains a single DNA molecule) and there is limited mitochondrial replication during preimplantation embryo development [Junsen, 1998; Piko, 1976], it is possible to determine relative mitochondrial copy number based on quantitative DNA amplification of the mitochondrial genome. Individual oocytes (of all studied genotypes) will be placed in 2.5 μl of PBS and stored at −70° C. DNA will be extracted as previously described [Dean, 2003]. ⅕ of the volume of the lysate will be used as a template for the PCR reaction using primers spanning the conserved region in the mitochondrial DNA. Primer sequences and starting conditions will be selected using Primer Express (ABI Prism) software and conditions will be determined as described in the above. All these experiments will be performed in the oocytes obtained from WT, WTC^{Cre}, Bcl-x^{del/fl} and Bcl-x^{fldel/Cre} females.

C. Metabolic effect of Bcl-x disruption.

Bcl-2 family members (Bcl-2 and Bcl-x) allow cells to maintain and/or improve oxidative phosphorylation and to adapt to changes in cellular metabolism [Vander Heiden, 2001, 2002]. This is particularly evident in somatic cells harboring mitochondrial mutations in which ATP production is decreased [Manfredi, 2003]. Since Bcl-x protein has been shown to facilitate efficient exchange of ADP for ATP in stressed cells, this molecule may support early preimplantation development under conditions of stress (in vitro culture in HTF) and perhaps maintain mitochondrial ATP production via coupling of TCA metabolism and oxidative phosphorylation after the switch from oxidation of pyruvate to the use of glucose as the main substrate [Martin, 1995; Gardner, 1986]. In this manner, Bcl-x may permit mitochondria to adapt to changes in metabolic demand.

Previous studies have shown that during development from a 2-cell embryo to a morula, embryos use pyruvate exclusively as their energy source and metabolize it via the TCA cycle. At the blastocyst stage, this metabolic pattern switches to predominantly anaerobic metabolism using glucose as the main energy source via glycolysis and the formation of lactate. The concentration of both glycolytic and TCA metabolites during this energetic switch in the murine preimplantation embryo have been measured and a profile of metabolites in normal embryos has been established. Stresses to the embryo such as elevated glucose concentrations (5.6 and 50 mM) or in vitro culture conditions lead to alterations in the normal metabolic pattern. Bcl-x may serve as a safety valve under these conditions to maintain mitochondrial ATP production via coupling of TCA metabolism and oxidative phosphorylation. Since it has been established that mammalian blastocysts are extremely sensitive to glucose deprivation [Chi, 2002; Moley, 1998], Bcl-x may be critical for maintenance of TCA flux under in vitro conditions and a deficiency of Bcl-x will compromise the ability of the blastocyst to respond to stress by increasing oxidative phosphorylation. These blastocysts will exhibit higher cell death indices.

The function of Bcl-x as a possible regulator of metabolic requirements in preimplantation embryos will be investigated by measuring metabolites of the TCA cycle as previously described [Chi, 2002; Chi, 2003]. Briefly, groups of embryos obtained from crosses described in A above will be subjected to metabolite microanalytic assays at the 2 cell, compacted 8 cell and blastocyst stages. Embryos of all studied genotypes will also be compared under favorable culture conditions (KSOM) or stressed conditions (KSOM supplemented with 50 mM glucose and HTF culture). The concentration of ATP, phosphocreatine (PCr), α-ketoglutarate, citrate, malate, fumarate, glutamate, pyruvate and fructose 1,6 bisphosphate (FBP) will be determined and compared among the different genotypes.

It is expected the embryos conceived from oocytes lacking Bcl-x will exhibit an increased rate of embryonic arrest and/or elevated cell death. This may be due to abnormal mitochondrial endowment, abnormal function or altered metabolic activity. Thus, a decrease in mitochondrial activity, mitochondrial copy number and/or altered subcellular mitochondrial distribution may be observed. Bcl-x null embryos may experience elevated levels of malate, fumarate, α-ketoglutarate, and glutamate, accompanied by lower levels of phosphocreatine and normal ATP levels, suggesting depletion of energetic stores with buffering of PCR. In addition, flux via the glycolytic pathway would also be compromised as evidenced by elevated FBP levels and lower pyruvate levels. Rescue experiments of the Bcl-x KO phenotype will be attempted by microinjection of recombinant Bcl-x protein into these embryos to see if the mitochondrial and metabolic changes revert to normal. The only unexpected outcome of these experiments is the lack of a preimplantation phenotype in embryos obtained from oocytes maternally lacking Bcl-x protein. In this case, the analysis will focus on Mcl-1 knockout embryos, as these have bona fide embryonic arrest during preimplantation development [Rinkenberger, 2000].

### Example 4

Establish if developmental competence can be enhanced by microinjection of recombinant Bcl-x or Mcl-1 proteins.

A. Determine the most efficient dose and protein isoforms of Bcl-x and/or Mcl-1 capable of rescuing abnormal preimplantation development in ICR mice.

Mice of outbred genetic background (ICR) exhibit a high rate of embryo arrest and increased cell death when cultured in HTF medium (FIG. 3). This model will be used as a screen to determine which recombinant protein and what dose is the most efficient in supporting preimplantation development under these mildly adverse conditions. As indicated in the preliminary results described herein, microinjection of recombinant Bcl-x protein, (Bcl-xΔC), facilitates preimplantation embryo development, leading to an increased rate of blastocyst formation and improved blastocyst quality. These results are consistent with the observation that culturing in HTF medium results in decreased Bcl-x and Mcl-1 expression. In addition, Rinkenberger et al [2000] have shown that Mcl-1 disruption leads to embryonic arrest.

Zygotes obtained from superovulated females 24 hours after hCG administration will be stripped of their cumulus cells, placed in HTF medium and injected with different amounts of commercially available (www.Bioclon.com) recombinant full length Bcl-x or Mcl-1 proteins, or a cocktail of both proteins. These injections will be compared to the Bcl-xΔC that lacks the C terminus or Bcl-xES [Schmitt, 2004] lacking the BH3/BH1 region. The importance of injection location (e.g. cytoplasmic versus pronuclear microinjection) will also be examined. Doses between 0.5-5 μg/μL in a volume of no more than 1 pL (pronuclear) or 5 pL (cytoplasmic) will be injected. Three control groups of zygotes will be either unmanipulated, buffer injected, or BSA injected (to control for a non-specific effect of increased protein content). In vitro developmental competence, frequency of cell death, markers of mitochondrial activity, and allocation of cells (by cell counts as described in Example 3A) to the ICM or TE lineage. Abnormalities in morphology, arrest, fragmentation and developmental delay will be further investigated by techniques described in Example 3 with at least 25 embryos per group per assay. This model will be used to determine the optimal amount and type of protein to inject for the best embryo development.

B. Are embryos obtained from the microinjection of recombinant proteins normal.

The object of this study is to determine whether embryos originating from recombinant protein injection develop any physiological anomalies. Pups will be created through embryo transfer using the information obtained in A above, (e.g., protein type, dose and intracellular location of delivery based on in vitro viability of injected embryos). The embryos will be injected and cultured as described above until the blastocyst stage (d3.5) at which time they will be transferred into pseudopregnant females of the ICR background. Control blastocysts will be assessed from unmanipulated HTF cultured embryos, buffer injected embryos, and recombinant protein injected embryos. As 10 embryos can be transferred per pseudopregnant female, a minimum of 10 pseudopregnant females/treatment/group will be required in order to generate the required number of animals, based on estimates that implantation rates will be reduced to ˜30% in buffer injected and unmanipulated HTF cultured zygotes.

After performing the outlined experiments, the physiological performance of the offspring will be analyzed. 10 mice per group per sex, equaling 60 mice total, will be tested. Phenotypic screens are used that are designed to identify defects in major physiologic systems including cardiovascular, renal, metabolic, hematopoietic, neurological and skeletal deficiencies in live mice.

C. Can recombinant Bcl-2 proteins improve human preimplantation embryo development?

Upon successful completion of the animal studies described above, a preliminary clinical trial will be initiated. Twenty patients who have undergone two cycles of IVF and who produce only very fragmented embryos (Grade 4 or 5), or embryos with delayed development (5 cells or less at 72 hours post fertilization), will be recruited (following standard procedures) for this pilot study. Based on the data accumulated in A above, patients of advanced chronological age (more than 40 year of age) will be considered, as these patients frequently produce abnormal embryos (both fragmented and arrested). Ovulation induction, oocyte retrieval, and in vitro fertilization will be performed using standard procedures as previously described [Casper, 1996; Sun, 1997]. Oocytes from each patient will be divided into two groups. Oocytes in group one will be injected with a single sperm in the usual ICSI procedure. Oocytes in group 2 will be injected with a single sperm aspirated into the injection pipette together with the most efficient recombinant protein as determined in the previous experiments. The volume for injection including both sperm and recombinant protein will be no more than 5 pL. Following injection, oocytes will be transferred into a 25 μl droplet of HTF medium supplemented with 5% human serum albumin in a plastic 60×15 mm petri dish, covered with mineral oil and incubated in a humidified 5% CO₂ 90% N₂ environment at 37° C. Cultured oocytes will be assessed for the presence of two pronuclei, indicative of normal fertilization at 16-18 h after ICSI and transferred into Global medium. The embryo score (cell number X 1/grade) will be determined for each embryo at 48 and 72 hours. Developmental progress will be followed up to 5 days in vitro, at which time morphologically normal appearing expanded blastocysts will be transferred into the uterine cavity. If normal embryo development occurs in any of the control oocytes, they will be transferred preferentially. However, as this group of patients has been selected for their embryonic abnormalities, this is unlikely. The pregnancies obtained from recombinant protein injection will be followed closely and the patients advised to consider amniocentesis to rule out chromosomal abnormalities. Babies born as a result of this procedure will be followed with assessment for normal development at birth, and at intervals thereafter.

It is anticipated that both Bcl-x and Mcl-1 will be capable of enhancing preimplantation embryo development. The extent of enhancement may differ since it is possible that Mcl-1 may be downstream of Bcl-x in preimplantation embryos (See FIG. 1). Cytoplasmic recombinant protein injection is also expected to be efficacious. In the alternative, routine IVF followed the next day by recombinant protein injection into the pronucleus of the fertilized zygote, as in the preliminary animal studies will be performed. As Mcl-1 is more labile (half time is only 6 h), a more stable mutant form of this protein may be desirable, as several ubiquitination sites have been mapped and shown to facilitate degradation of Mcl-1. The regions of Bcl-x and/or Mcl-1 responsible for the observed positive influence on embryo development will be examined via deletion of BH domains or the transmembrane mitochondrial region. Other phosphorylation or caspase cleavage mutants that have recently been described will also be investigated [Grethe, 2004; Domina, 2000; Domina, 2004; Clohessy, 2004; and Michels, 2004].

Clinical Significance:

Preimplantation embryo arrest and fragmentation as a result of programmed cell death is common in assisted reproductive technologies (ART), especially with reproductive ageing. Injection of ooplasm from healthy donor eggs, or of mitochondria from fetal cells, both appear to enhance mitochondrial function and improve embryo developmental competence. However, legislation in some jurisdictions does not make it possible to take advantage of the positive effects of ooplasmic transfer or mitochondrial injections as both result in mitochondrial heteroplasmy in the developing embryo. Moreover, recent findings of altered renal function in mice carrying neutral mitochondrial heteroplasmy suggest a potential negative impact on overall health. Injection of recombinant proteins of the Bcl-2 family will make it possible to achieve the benefits of mitochondrial enhancement without altering the fetal genome. In addition, these recombinant proteins are stable, can be lyophilized and reconstituted at the time of injection, and can be combined with mechanical sperm injection, resulting in a clinically feasible, widely available treatment requiring no extra equipment or skills apart from those needed for ICSI. This new procedure could result in a significant improvement in clinical pregnancy outcome in ART.

### Example 5

### SUMMARY

Using germ cells from highly inbred mouse strains, herein two prominent genetic modifiers of apoptosis were uncovered. The first, present in AKR/J mice, causes genomic instability. This is reflected by numerous DNA double-strand breaks in freshly isolated cells, leading to a rapid onset of apoptosis that can be reversed by microinjection of recombinant Rad51 protein. The second is manifested in FVB mice by mitochondrial dimorphisms and frequent outer mitochondrial membrane breaks. This phenotype is correlated with enhanced cytochrome c release and high apoptosis susceptibility, the latter of which is suppressed by pyruvate treatment, Smac/DIABLO deficiency, or microinjection of “normal” mitochondria. The results of this study identify the existence of genetic modifiers of genomic integrity and mitochondrial ultrastucture that profoundly influence cell death in mammals.

Materials and Methods

The following Materials and Methods were employed in the study disclosed in this Example.

Materials and Methods

Animals: Wild-type AKR/J (Jackson Laboratories, Bar Harbor, Me.), FVB (Taconic, Germantown, N.Y.), C57BL/6 (Charles River Laboratories, Wilmington, Mass.) and B6C3F1 (Charles River Laboratories, Wilmington, Mass. or Saint-Constant, Quebec, Canada) mice between 2-4 months of age were purchased for this study. In some experiments, mutant mice lacking Smac/DIABLO (Okada et al. 2002) were outcrossed 5 generations onto a FVB genetic background for analysis. All experiments involving animals described herein were reviewed and approved by the institutional animal care and use committees of Massachusetts General Hospital, Michigan State University, and Mount Sinai Hospital.

Oocyte isolation and culture: Oocytes were collected after superovulation as described (Perez et al. 1997; Perez et al. 1999; Morita et al. 2000). Briefly, ovulated oocytes were denuded of cumulus cells by a 1-min incubation in 80 IU ml of hyaluronidase (Sigma, St. Louis, Mo.), followed by three washes with culture medium. All cultures were carried out in human tubal fluid (Irvine Scientific, Santa Ana, Calif.) supplemented with 0.5% BSA. The oocytes were maintained in 0.1 ml drops of culture medium under paraffin oil, and incubated at 37° C. in a humidified atmosphere of 5% CO₂ and 95% air.

In-vitrofertilization and embryo culture: Female mice were superovulated as described above and cumulus-oocyte complexes from the indicated strains were mixed with capacitated sperm collected from adult male mice of the respective strain for in-vitro fertilization (Morita et al. 2000). After 2 h, the cumulus-oocyte complexes were washed and maintained in KSOM medium (Specialty Media, Phillipsburg, N.J.) to determine fertilization rates and monitor embryonic progression. Since zygotes progress to the blastocyst stage of development within 96 h and begin hatching, embryos were fixed at this time and stained with the DNA-binding dye Hoechst 33342 (Sigma) for light and fluorescence microscopic analysis of blastocyst cell number and quality. Apoptosis analyses: Oocytes were evaluated as described (Perez et al. 1997; Perez et al. 1999; Mortia et al. 2000) for characteristics of apoptosis, including morphological changes (e.g., cellular condensation, budding and fragmentation) and biochemical alterations (i.e., DNA cleavage using Comet Assay Kit; Trevigen, Gaithersburg, Md.). Analysis of DNA “comets” to generate quantitative data on the percent of undamaged versus damaged DNA was performed using the VisComet program (Impuls Computergestutzte Bildanalyse GmbH, Gilching, Germany). Assessment of MMP: Oocytes were stained using the membrane sensitive dye JC-1 (DePsipher™; R&D Systems, Minneapolis, Minn.) and then viewed by deconvolution microscopy (Olympus IX70) with fluorescein isothiocyanate (FITC) and rhodamine isothiocyanate (RITC) filters (Acton et al. 2004). For each oocyte, ten optically sectioned (2 μm thick) images were captured and analyzed using Delta Vision Software (Applied Precision, LLC., Issaquah, Wash.) to quantitate fluorescence signal intensity. After subtraction of background noise, the ratio of RITC (J-aggregate) to FITC (J-monomer) was determined for each section, and an average ratio of J-aggregate to J-monomer for the entire oocyte was calculated.

Measurement of ATP: The ATP Bioluminescent Somatic Cell Assay Kit (Sigma, Oakville, Ontario, Canada) was used to assess ATP content in groups of 25 oocytes following extrapolation from a standard curve composed of 10 ATP concentrations ranging between 18 pmol and 7.2 μmol per sample volume. Duplicate luminometer readings were taken from each sample over 20 sec intervals, and the average relative light unit readings were used to determine ATP content in the samples against the standard curve.

Bioreduction assay: Oocyte reduction potential was assessed by use of 3-(4,5-dimethylthiazol-2-yl) -2,5-diphenyl-tetrazolium bromide (MTT), a water-soluble tetrazolium salt that precipitates as a colored formazan upon reduction (Bernas and Dobrucki 2002). Groups of 25 oocytes were cultured for 4 h in 1.2 mM MTT and then washed several times in phenol-free RPMI medium 1640 (Sigma) supplemented with 0.5% BSA. The oocytes were then transferred into 100 μl of dimethylsulfoxide (DMSO, Sigma) in 96-well plates, and the intensity of the precipitated formazan product was determined using an uQuant Plate Reader and KC Junior Software (Bio-Tek Instruments, Winooski, Vt.).

Reduced glutathione (GSH) content: Quantitation of cellular GSH was assayed by use of monochlorobimane (Molecular Probes, Eugene, Oreg.), a cell-permeant dye that becomes fluorescent upon conjugation to thiol groups (Nasr-Esfahani and Johnson 1992). One group of untreated oocytes served as a control for measuring baseline GSH levels, whereas two other groups of oocytes were treated with I mM hydrogen peroxide (Sigma). For the latter, one group was exposed to hydrogen peroxide for 90 min (oxidative insult) whereas the second group was incubated with hydrogen peroxide for 30 min and transferred to fresh culture medium without hydrogen peroxide for an additional 60 min (recovery after oxidative insult). During the last 15 min of incubation, monochlorobimane was added to a final concentration of 1 mM, after which the oocytes were washed in fresh culture medium. Imaging and micrograph analyses were then conducted using a deconvolution microscope equipped with a cyan fluorescent protein filter. The average fluorescence intensities were tabulated on a per oocyte basis, and the data were then compiled per group. Reactive oxygen species: Formation of ROS was determined through the use of 2′,7′-dichlorodihydrofluorescein diacetate (H₂DCFDA; Molecular Probes) as described (Yang et al. 1998). The H₂DCFDA dye is membrane permeant, and upon entering the cell the acetate groups are hydrolyzed, creating a membrane impermeant form of the dye (H₂DCF). Endogenous ROS oxidize this polar form of the dye to a quantifiable fluorogenic compound (DCF). Oocytes were mixed with a freshly prepared solution of 0.01 M H₂DCFDA and incubated for 15 min. After extensive washing in fresh culture medium, imaging was carried out using a deconvolution microscope with a FITC filter. Total light intensity for each optical section and average light intensity for each oocyte were determined using the Delta Vision Software Analysis program. In order to determine baseline fluorescence, control oocytes (unstained) were incubated with an appropriate volume of vehicle (DMSO) prior to imaging. In some experiments, oocytes were cultured without or with pyruvate (10 mM; Sigma) for 6 h prior to being imaged for ROS content. In addition, the following inhibitors of the oxidative phosphorylation chain were used to assess specificity for mitochondrial ROS production: rotenone (inhibits Complex I activity), antimycin-A (inhibits Complex III activity) or oligomycin (inhibits Complex V activity). Each inhibitor (2 μg ml⁻¹) was individually added and the cultures were continued for 30 min prior to analysis of ROS content. Somatic cell cultures: Ovarian somatic (granulosa) cells were isolated as described (Matikainen et al. 2001). Briefly, immature (21-24 days postpartum) FVB and B6C3F1 female mice were injected with 10 IU of equine chorionic gonadotropin, and ovaries were removed 42 h later. The stimulated follicles were punctured with fine needles to collect granulosa cells into Waymouth's MB752/1 medium (Life Technologies) supplemented with penicillin, streptomycin and L-glutamine. After trypan blue staining, approximately 1×10⁶ viable cells were seeded and cultured for 72 h in 100-mm dishes containing 10 ml of culture medium supplemented with 10% FBS (HyClone Laboratories, Logan, Utah). Mouse embryonic stem cells (line R1 derived from a 129 genetic background) were seeded at a density of approximately 2×10⁶ cells per 0.1 % gelatin-coated 100-mm plate, cultured under standard conditions in a humidified incubator at 37° C. under 5% CO₂ and passaged when the cultures reach 80% confluency.

Isolation of mitochondria: When the somatic cell cultures reached 80% confluency, 2 ml of mitochondrial lysis buffer (0.3 M sucrose, 1 mM EDTA, 5 mM MOPS, 5 mM KH₂PO₄, 0.1% BSA) were added to each plate, and the cells were removed using a cell scraper. The cell suspension was transferred into a small glass tissue bouncer and homogenized until smooth (approximately 10 up and down strokes) and the lysate was centrifuged at 600×g for 30 min at 4° C. The supernatant was removed and spun at 10,000×g for 12 min at 4° C., and the resulting crude mitochondrial pellet was resuspended in 0.2 ml of 0.25 M sucrose. This sample was then layered over a 25-60% Percoll density gradient diluted with 0.25 M sucrose and centrifuged at 40,000×g for 20 min at 17° C. The interface band was extracted from the gradient and washed in 2 volumes of 0.25 M sucrose prior to a final centrifugation at 14,000×g for 10 min at 4° C. to yield a mitochondrial pellet, as described (Darley-Usmar et al. 1987).

Oocyte microinjection: Microinjection needles and holding pipettes were made using a Sutter puller (Sutter Instruments, Novato, Calif.) and a De Fonbrune Microforge (EB Sciences, East Granby, Conn.). The microinjection needles had inner diameters of 5 μm with blunt tips. The experimental material to be injected or its negative control (mitochondria or sucrose, recombinant Rad51 or BSA, cytochrome c or cytochrome b, recombinant Smac/DIABLO or BSA, respectively) was aspirated into the needle by negative suction. The mitochondrial suspension in sucrose (5-7 pl containing approximately 1×10³ or 5×10³ mitochondria from embryonic stem cells or granulosa cells, respectively), recombinant Rad51 (Kurumizaka et al. 1999; 6 pl of a 3.6 μg μl⁻¹ stock per oocyte), cytochrome c (6 pl of a 400 μM stock per oocyte) or recombinant Smac/DIABLO (Du et al. 2000; 6 pl of a 700 μg μl⁻¹ stock) were injected into oocytes using a Piezo micromanipulator. Oocytes that did not survive the microinjection procedure (routinely less than 25%) were discarded, and the remaining oocytes were transferred for culture and assessment of apoptosis.

Electron microscopy: Oocytes were cultured without or with pyruvate (10 mM) for 3 h prior to being fixed for EM tomography. The oocytes were embedded in agarose and prepared for tomography using conventional protocols for good structural preservation (Ricci et al. 2004). To survey the preservation quality of the oocytes, thin-sectioned materials (−80 nm) were examined using a JEOL 1200FX electron microscope. Three-dimensional reconstructions of portions of the cell containing mitochondria were generated using standard techniques (Perkins et al. 2003). Sections with a thickness between 300-500 nm were cut out, and stained for 30 min in 2% aqueous uranyl acetate, followed by 30 min in lead salts. Next, fiducial cues consisting of 20 nm colloidal gold particles were deposited on both sides of each section.

For each reconstruction, a series of images at regular tilt increments were collected with a JEOL 4000EX intermediate-voltage electron microscope operated at 400 kV. In order to limit anisotropic specimen thinning during image collection, the specimens were irradiated before initiating a tilt series. Pre-irradiation in this manner subjected the specimen to the steepest portion of the non-linear shrinkage profile before images were collected using a slow-scan CCD camera with 1960×2560 pixels at a resolution of 1.1 nm. Tilt series were recorded at a magnification of 20,000× with an angular increment of 2° from −60° to +60° about an axis perpendicular to the optical axis of the microscope using a computer-controlled goniometer to achieve accurate increments at each angular step. Illumination was held to near parallel beam conditions and optical density maintained constant by varying the exposure time. The IMOD package (Mastronarde 1997) was used for alignment and the TxBR package (National Center for Microscopy and Imaging Research, San Diego, Calif.) was used for generating the reconstructions.

Volume segmentation was performed by manual tracing in the planes of highest resolution with the program Xvoxtrace (Perkins et al. 1997a; Perkins et al. 1997b). Mitochondrial reconstructions were visualized using Analyze (Mayo Foundation, Rochester, Minn.) or Synu (National Center for Microscopy and Imaging Research) as described (Perkins et al. 2001). These programs allow one to step through reconstructed slices in any orientation and to track or model features of interest in three dimensions. Measurements of structural features were made within segmented volumes by the programs, Synusurface and Synuvolume (National Center for Microscopy and Imaging Research). Quantitation of the presence of different mitochondrial structures was performed on random transmission EM-acquired images.

Cytochrome c release: Oocyte mitochondrial enriched fractions were prepared by differential centrifugation as described above. In some experiments, the oocytes were pre-incubated without or with pyruvate (10 mM) for 2 h. Cytochrome c release was evaluated by a sensitive and specific immunoassay, using a commercial ELISA kit (Quantikine^{R}M assay; R & D Systems) according to the manufacturer's instructions. The light emitted was quantified by using a microtiter plate reader at 450 nm, and translated into cytochrome c concentrations through a standard curve.

Data presentation and statistical analysis: All experiments were independently replicated at least three times with different mice. Combined data from the replicate experiments were subjected to a one-way analysis of variance followed by Scheffe's F-test, Student's t-test or chi-square analysis. P values less than 0.05 were considered statistically significant. Graphs represent the mean (±SEM) of combined data from the replicate experiments, whereas representative photomicrographs of DNA damage and EM-based analyses of mitochondria are presented.

Results

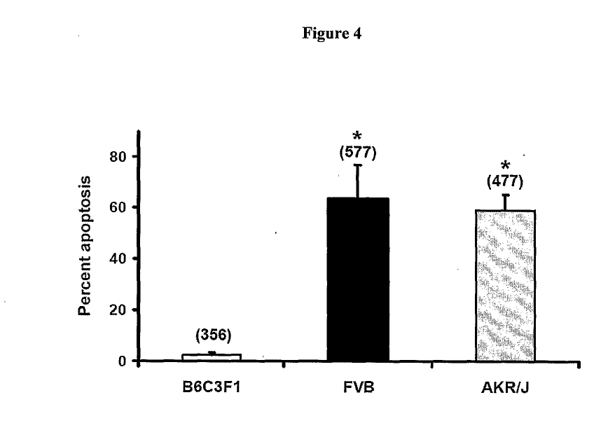

Apoptosis susceptibility differs among genetic strains: Mature germ cells (oocytes) obtained by superovulation of adult female mice undergo apoptosis when maintained in vitro (Perez et al. 1997; Perez et al. 1999). The initiation of apoptosis in these cells occurs through a well-defined genetic program involving Bcl-2 family members and caspases (Tilly 2001). During the course of the studies of female germ cell death over the past few years, considerable variability has been noted in the extent of apoptosis in oocytes collected from different inbred strains of mice following in-vitro culture. Of the strains evaluated, oocytes from AKR/J and FVB mice exhibited the highest susceptibility to apoptosis when compared with the relatively low level of death observed in oocytes collected from either C57BL/6 mice (Perez et al. 1997; Perez et al. 1999) or B6C3F1 mice (FIG. 4). Moreover, the high incidence of apoptosis in FVB oocytes cultured in vitro for 24 h (FIG. 4) was found to closely match that observed in FVB oocytes collected in vivo 24 h after ovulation (81±9%; mean ±SEM, n=104 oocytes).

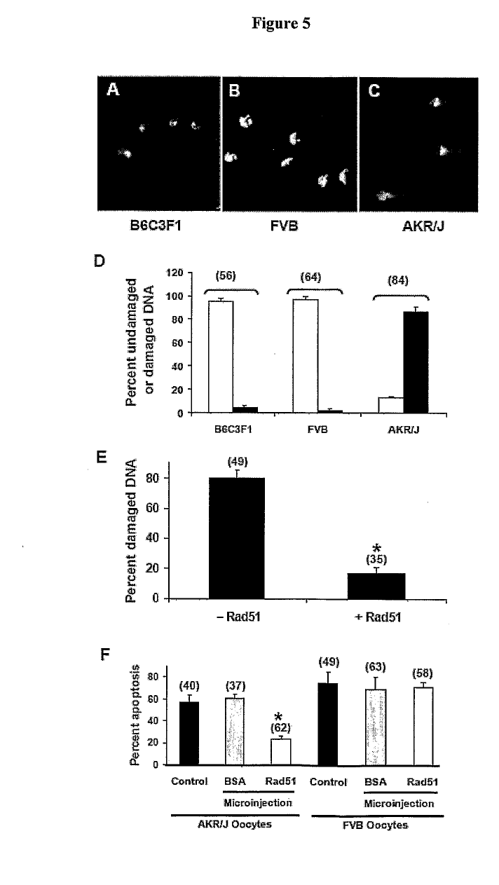

Faulty DNA damage repair elevates germline apoptosis susceptibility in AKR/Jmice: To identify the mechanisms responsible for the strain-dependent variability in germ cell death, DNA integrity was first assessed in oocytes of the various strains given that DNA damage is one of the most well characterized triggers of apoptosis (Li and Zou 2005). Chromosomal DNA integrity within freshly isolated AKR/J oocytes was compromised as evidenced by a high degree of preexistent damage (FIGS. 5C, D), and the percent of damaged DNA in AKR/J oocytes remained high after 6 h of culture (80±6%; mean ±SEM, n=49 oocytes). In contrast, B6C3F1 and FVB oocytes harbored mostly intact DNA irrespective of whether the analyses were performed using freshly isolated (FIGS. 5A, B, D) or cultured (data not shown) oocytes.

To determine if the preexistent DNA damage in freshly isolated AKR/J oocytes was causally related to their enhanced apoptosis susceptibility, the ability of a DNA repair protein to rescue the phenotype was next assessed. Microinjection of recombinant Rad51 protein into AKR/J oocytes decreased the extent of DNA damage over a subsequent 6 h culture period when compared with non-injected AKR/J oocytes cultured in parallel (FIG. 5E). In addition, Rad51 microinjection significantly suppressed apoptosis in AKR/J oocytes compared to both non-injected and bovine serum albumin (BSA)-injected AKR/J oocytes (FIG. 5F). By comparison, microinjection of Rad51 had no effect on the high incidence of apoptosis in FVB oocytes maintained for 24 h in vitro (FIG. 5F).

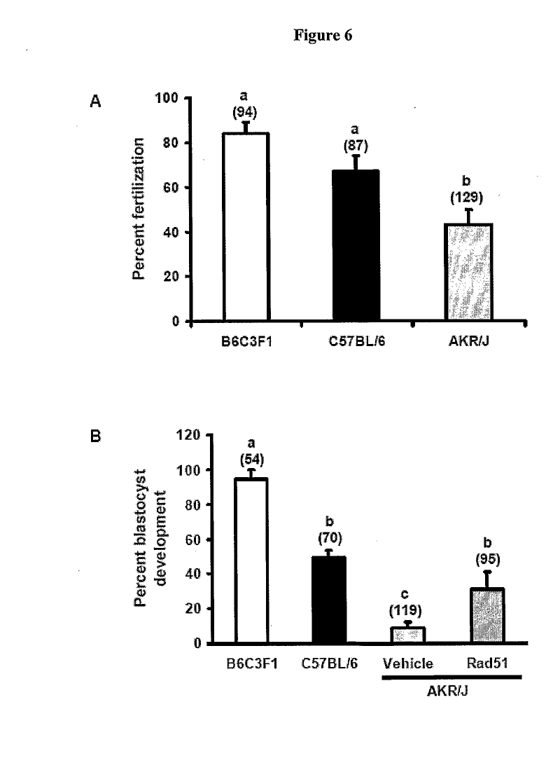

Rad51 reverses the reduced embryonic developmental capacity in AKR/J mice: Female AKR/J mice have fewer litters and reduced litter sizes compared with other commonly studied mouse strains (see Festing Mouse Genome Informatics at http://www.informatics jax.org/). To determine if the defective DNA repair, and the resultant enhancement of apoptosis, observed in AKR/J germ cells contributes to the inferior reproductive performance associated with this genetic background, the competency of AKR/J oocytes to be fertilized and proceed through preimplantation embryonic development was assessed. In-vitro fertilization rates using AKR/J oocytes were significantly lower than those observed using either B6C3F1 or CS7BL/6 oocytes as controls (FIG. 6A). Moreover, once fertilized, 94% of the B6C3F1 zygotes and 49% of the CSLBL/6 zygotes were competent to reach the blastocyst stage (FIG. 6B). In contrast, less than 10% of the fertilized AKR/J oocytes were able to complete blastocyst development (FIG. 6B). However, microinjection of Rad51 into fertilized AKR/J oocytes resulted in a pronounced rescue of this defect in that more than 30% of the microinjected AKR/J zygotes successfully completed preimplantation embryonic development to the blastocyst stage (FIG. 6B).

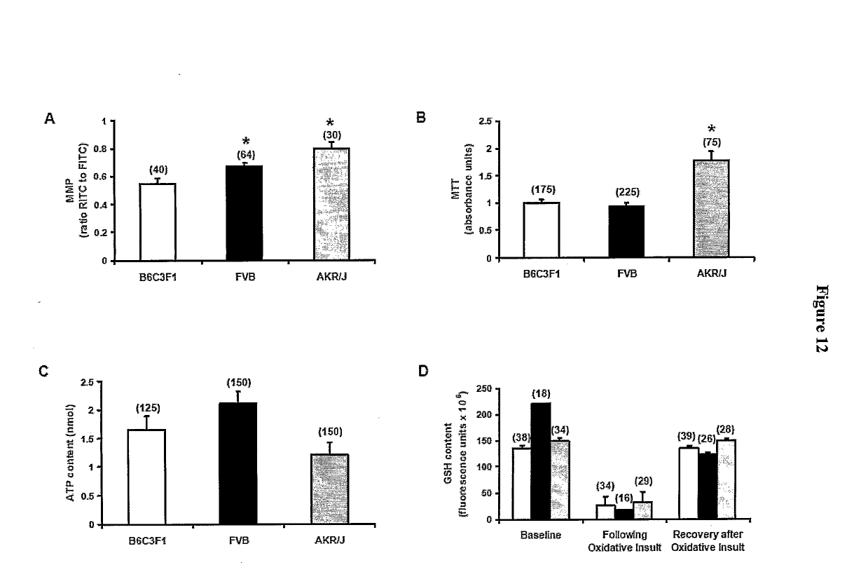

Strain-dependent differences in mitochondrial metabolic parameters: The absence of DNA damage and the inability of Rad51 microinjection to suppress apoptosis in FVB oocytes suggested that the enhanced cell death susceptibility observed in germ cells of this genetic background was due to a modifier locus unrelated to DNA integrity. In light of the wealth of information available on the importance of mitochondria to the survival and death of cells (Danial and Korsmeyer 2004; Green and Kroemer 2004), experiments were next conducted to analyze mitochondrial function in oocytes of the various strains. In the first set of studies the mitochondrial membrane potential (MMP) in oocytes of both ‘apoptosis-prone’ strains (FVB and AKR/J) was slightly elevated when compared with the MMP in B6C3F1 oocytes (FIG. 12A). An increased level of mitochondrial activity was also detected in AKR/J, but not FVB, oocytes when compared to B6C3F1 oocytes using the MTT bioreduction assay (FIG. 12B). However, no significant strain-dependent differences in oocyte ATP content (FIG. 12C) or glutathione content (baseline, following oxidative insult, or recovery after oxidative insult; FIG. 12D) were noted.

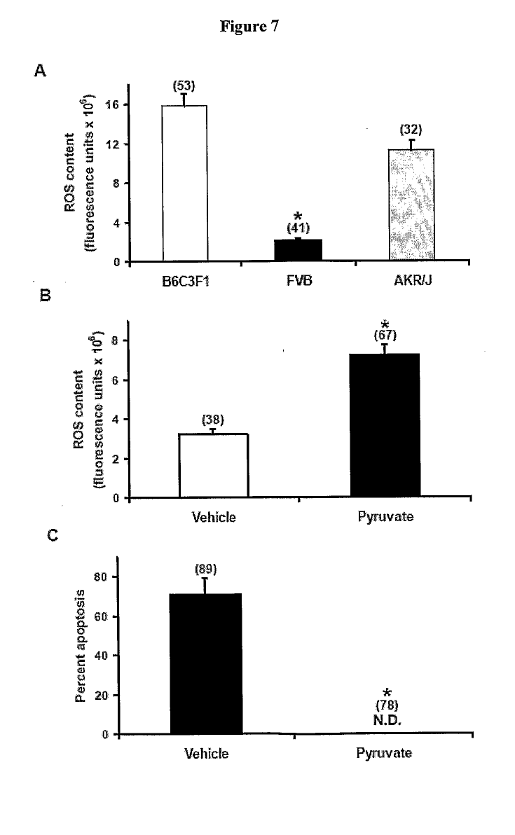

The levels of reactive oxygen species (ROS) were next assessed in oocytes of the three strains since ROS are natural by-products of oxidative phosphorylation and thus reflect the metabolic activity of mitochondria. In freshly isolated oocytes, no significant differences in ROS content were found between AKR/J and B6C3F1 oocytes. However, ROS content in freshly isolated FVB oocytes was less than 20% of that found in B6C3F I or AKR/J oocytes (FIG. 7A). Due to the low level of ROS detected in FVB oocytes, it was postulated that their mitochondrial metabolic function was impaired. Since oocytes do not rely on glycolysis for energy production but instead favor pyruvate as substrate for oxidation (Downs and Hudson 2000), the effects of pyruvate supplementation on oocyte metabolic performance and survival were explored. Treatment of FVB oocytes for 6 h with 10 mM pyruvate more than doubled ROS content in comparison to untreated FVB oocytes (FIG. 7B). Moreover, the presence of pyruvate completely suppressed apoptosis in FVB oocytes maintained for 24 h in vitro (FIG. 7C). Using inhibitors of the mitochondrial electron transport chain, mitochondria were confirmed to be the primary source of the ROS in that co-treatment of FVB oocytes with rotenone reduced ROS content by 54% while treatment with antimycin-A or oligomycin reduced ROS content by 70% (data not shown).

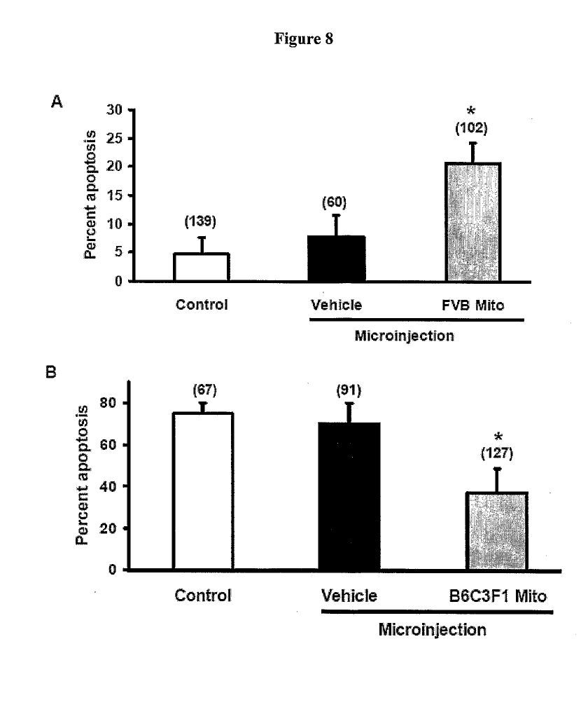

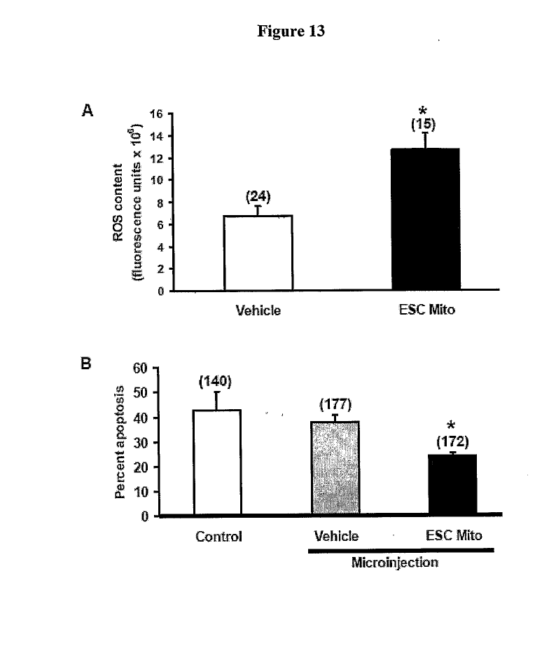

Mitochondrial microinjection reduces the high apoptosis susceptibility in FVB oocytes: To test if defective mitochondrial function in FVB oocytes directly contributes to the enhanced apoptosis susceptibility observed in these cells, mitochondria collected from FVB mice were microinjected into B6C3F1 oocytes and the incidence of apoptosis over a subsequent 24 h culture period was recorded. Microinjection of vehicle alone did not affect the low basal rate of apoptosis seen in oocytes of this strain; however, microinjection of FVB mitochondria into B6C3F1 oocytes significantly increased apoptosis when compared with those levels observed in non-injected or vehicle-injected B6C3F1 oocytes (FIG. 8A). Conversely, microinjection of mitochondria collected from B6C3F I mice into FVB oocytes reduced apoptosis by approximately 50% compared with non-injected or vehicle-injected FVB oocytes (FIG. 8B). In parallel experiments, microinjection of mitochondria derived from mouse embryonic stem cells, which possess mitochondria that are more similar to those found in germ cells, also reduced the extent of apoptosis in FVB oocytes cultured for 24 h (FIG. 13).

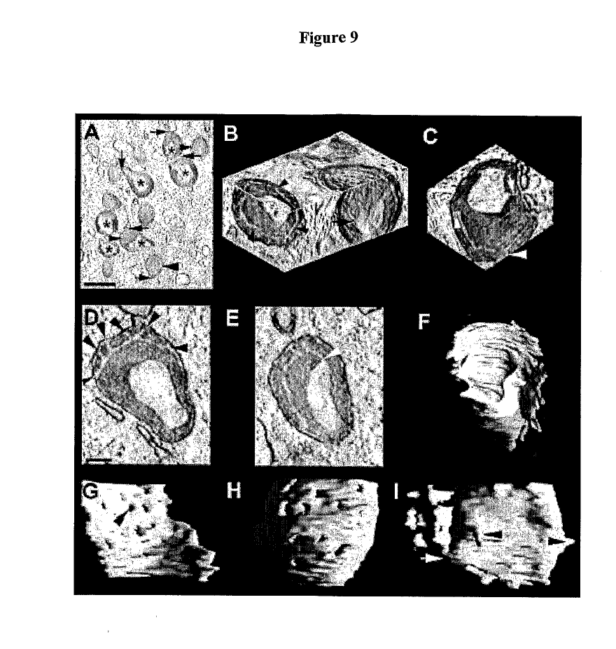

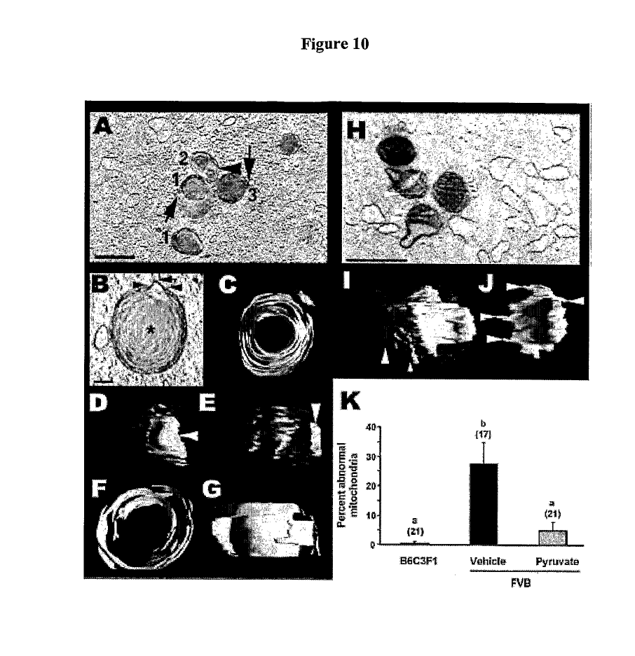

Electron microscopic tomography reveals structural anomalies in FVB mitochondria: To gain additional insight into the properties of FVB mitochondria that may be involved in elevating apoptosis susceptibility, the 3-dimensional architecture of individual mitochondria in FVB oocytes was reconstructed using electron microscopic (EM) tomography and compared with that of B6C3F1 oocyte mitochondria. Mitochondria were found distributed in small clusters in both apoptosis-resistant B6C3F1 oocytes (FIG. 9A) and apoptosis-prone FVB oocytes (FIG. 10A). Additionally, contacts between mitochondria and the endoplasmic reticulum were often observed in oocytes of both strains (FIG. 9A; FIG. 10A, B). Despite these similarities, striking differences in the ultrastructural features of mitochondria in B6C3F1 and FVB oocytes were uncovered. Mitochondria in B6C3F1 oocytes typically displayed a dichotomy of cristae structure, in which one-half appeared as a single, large cristal compartment. The other half consisted of one to four lamellar compartments, with one of the lamellar cristae consistently located around the periphery of the mitochondrion (FIG. 9A-E). Crista junctions were commonly observed in these mitochondria as well.

By comparison, mitochondria present in FVB oocytes exhibited a number of degenerative features not observed in mitochondria of B6C3F1 oocytes. For example, the large cristal compartment that was so prominent in the mitochondria of B6C3F1 oocytes (FIG. 9) was noticeably absent in many of the mitochondria in FVB oocytes (FIG. 10A, B). In its place was found a centrally located matrix. Another prominent difference was that the peripheral cristae in mitochondria of FVB oocytes had transformed into “onion-like” whorls (FIGS. 10B, C). Moreover, crista junctions were no longer discernable, and it was common to find ruptured outer mitochondrial membranes that allowed the inner boundary membranes to extend outward (FIG. 10A-G). Other mitochondria in FVB oocytes possessed vacuolated cristae but these cristae were adorned with abnormal internal “blobs”, possibly representing orphaned satellite volumes of the matrix (FIG. 10A).

Since pyruvate was found in earlier experiments to reverse the high apoptosis-susceptibility in FVB oocytes (FIG. 7C), it was next explored if pyruvate exerted its effects at the level of mitochondrial ultrastructure. In FVB oocytes exposed for 3 h to 10 mM pyruvate, the majority of mitochondria exhibited features similar to those of mitochondria in B6C3F1 oocytes (FIG. 10H, I). The large cristal compartment, and both the transverse and peripheral cristae, were present in the same dichotomy, and crista junctions were prominent (FIG. 10H-J). Scoring mitochondria with abnormal morphology in random sections of B6C3F1 oocytes as well as in untreated and pyruvate-treated FVB oocytes revealed that pyruvate reduced the number of abnormal mitochondria in FVB oocytes by more than 75% compared with FVB oocytes exposed to vehicle (FIG. 10K).

Ultrastructural anomalies in FVB mitochondria facilitate cytochrome c release: The presence of abnormally large perforations in the outer membranes of mitochondria in freshly-isolated FVB oocytes (FIG. 10C-G), and the ability of pyruvate to reduce both the number of abnormal mitochondria (FIG. 10K) and the onset of apoptosis (FIG. 7C) in FVB oocytes, suggested that the ultrastructural anomalies in mitochondria of oocytes from this strain directly contribute to their enhanced apoptosis susceptibility. In keeping with this, and past studies showing that mitochondrial cytochrome c release plays a central role in the execution of apoptosis (Danial and Korsmeyer 2004; Green and Kroemer 2004), it was found that during short term incubations mitochondria collected from FVB oocytes released 19% more cytochrome c (56±15% of the total mitochondrial cytochrome c pool present prior to incubation was released; mean ±SEM, n=4 experiments using 300 oocytes per experiment) than did mitochondria from B6C3F1 oocytes (37±8% of the total mitochondrial cytochrome c pool present prior to incubation was released; mean ±SEM, n=4 experiments using 300 oocytes per experiment). However, when mitochondria isolated from FVB oocytes were pre-incubated for 2 h with 10 mM pyruvate, there was a reduction in the amount of cytochrome c released during the subsequent incubation period (34±2% of the total mitochondrial cytochrome c pool present prior to incubation; mean ±SEM, n=4 experiments using 200 oocytes per experiment) to levels comparable to those released by mitochondria collected from B6C3F1 oocytes.

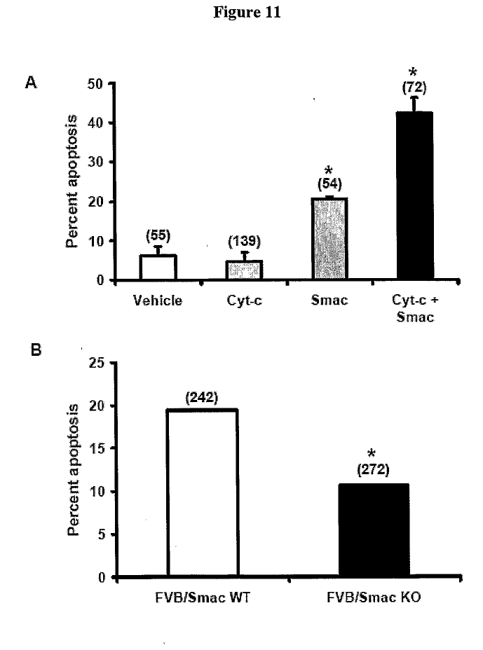

Cytochrome c and Smac/DIABLO synergize to promote germ cell apoptosis: In light of the findings above, additional experiments were conducted to determine if cytochrome c, when experimentally elevated in the cytoplasm of oocytes, could directly activate apoptosis. Microinjection of cytochrome c into B6C3F 1 oocytes, which have a low basal rate of apoptosis in vitro (FIG. 4), did not affect the incidence of apoptosis in a subsequent 24-h culture period (FIG. 11A). However, microinjection of recombinant Smac/DIABLO, a death-promoting protein released along with cytochrome c from mitochondria (Du et al. 2000; Verhagen et al. 2000), increased the incidence of apoptosis in cultured B6C3F1 oocytes by almost 3-fold. Moreover, microinjection of both cytochrome c and Smac/DIABLO further increased the incidence of apoptosis in B6C3F1 oocytes over that obtained using Smac/DIABLO alone (FIG. 11A). To directly test the in vivo significance of Smac/DIABLO to FVB oocyte death, mutant mice lacking functional Smac/DIABLO (Okada et al. 2002) were outcrossed onto a FVB genetic background and oocytes were collected for analysis. In keeping with the data obtained from the microinjection experiments, the incidence of apoptosis in Smac/DIABLO deficient FVB oocytes was reduced by almost one-half when compared with oocytes collected from wild-type female littermates and cultured in parallel (FIG. 11B).

Discussion

Experimental evidence demonstrating wide phenotypic variations in traits that are inherited in a Mendelian fashion has been available for nearly a century (Bridges 1919). While there are a number of reasons why the simple inheritance of a trait can be phenotypically variable—including alternative alleles and environmental factors—one of the most prominent is represented by genetic modifiers (Nadeau 2003). In humans, the role of modifier genes in disease predisposition has gained considerable attention in the past several years as modifier loci that affect the development of a wide array of health problems, including developmental malformations, cystic fibrosis, deafness and cancer, have been reported (Zielenski et al. 1999; Riazuddin et al. 2000; Dragani 2003; Slavotinek and Biesecker 2003). The majority of work on genetic modifiers in mammals has, however, originated from studies of inbred mouse strains (Nadeau 2003). Although in most cases the identities of the genes responsible for strain-dependent phenotypic modifications have yet to be determined, the impact of genetic background on the emergence or repression of a given phenotype in mice—especially those harboring targeted gene disruptions or ectopically expressed transgenes—has been known for years. In the context of cell death regulation, one of the more obvious examples of this comes from studies of mutant mice lacking the apoptotic executioner enzyme, caspase-3. When originally described, caspase-3 deficiency was reported to result in a perinatal lethal phenotype due primarily to excessive neural precursor cell expansion and exencephaly during development (Kuida et al. 1996). However, after backcrossing caspase-3 deficient mice onto a congenic C57BL/6 background, the brain phenotype was minimized and the perinatal lethality was lost, allowing the mutant animals to survive as adults (Leonard et al. 2002).

In the present work, two distinct strain-dependent modifiers that enhance apoptosis susceptibility have been identified. The first of these, observed in AKR/J mice, is manifest by an apparent inability of female germ cells to carry out repair of DDSBs. This conclusion is based on the finding that a high degree of preexistent DNA damage is present in freshly isolated AKR/J oocytes. Further, this phenotype could be minimized by microinjection of recombinant Rad51 into the oocytes, which was followed by a marked reduction in the incidence of apoptosis in these cells. The reduced DNA repair capacity seen in AKR/J germ cells was also associated with a severe impairment in preimplantation embryonic developmental competency following fertilization, which likely explains the poor reproductive outcomes previously reported for AKR/J mice (see Festing Mouse Genome Informatics at http://www.informatics.jax.org/). Perhaps even more striking, the life expectancy of AKR/J mice is 10 months or less under conventional housing conditions (see Festing Mouse Genome Informatics at http://www.informatics.jax.org/). This is considerably shorter than that of many other strains of mice, including FVB, C57BL/6 and B6C3F 1, which routinely live past 24 months of age under the same conditions (see Festing Mouse Genome Informatics at http://www.informatics.jax.org/). In addition, AKR/J mice exhibit an abnormally high predisposition for the development of leukemia (see Festing Mouse Genome Informatics at http://www.informatics.jax.org/). Although it is not known if the cancer predisposition or the early death of AKR/J mice is related to the molecular phenotype identified herein (i.e., inadequate DNA repair), such an outcome would be in keeping with past studies linking DNA instability and defective DNA repair to both cancer and aging in mammals (Charames and Bapat 2003; Lombard et al. 2005). Given the ability of Rad51 to reduce the extent of DNA damage and thus apoptosis susceptibility in AKR/J oocytes, as well as to dramatically improve the preimplantation embryonic developmental potential of AKR/J zygotes, ubiquitous overexpression of Rad51 using a transgenic approach could reduce the incidence of leukemia and/or extend longevity in AKR/J mice.

The second genetic modifier of apoptosis identified herein manifested as a striking mitochondrial defect in FVB mice, which is of particular interest in that transgenic mice are most frequently generated on this genetic background. Accordingly, phenotypes of transgenic lines produced using FVB zygotes for pronuclear injection—especially those involving the ectopic expression of apoptosis-regulatory genes—may reflect the outcome of a much more complex genetic interplay than simply the impact of the transgene being studied. In any case, there are at least three known mechanisms through which mitochondria can impact on the death susceptibility of cells: 1) disruption of electron transport, oxidative phosphorylation, and ATP production; 2) alteration of cellular reduction/oxidation potential; and 3) release of proteins into the cytoplasm that trigger apoptosis. In general, when compared with B6C3F1 or AKR/J oocytes, FVB oocytes did not demonstrate any marked changes in reduction/oxidation pathways as measured through MTT or GSH assays, nor did they exhibit significant differences in ATP levels. There was, however, a considerable reduction in ROS content in FVB oocytes, indicative of diminished metabolic activity and probably a disruption in the electron transport chain. Further to this, analysis of mitochondrial ultrastructure through EM tomography indicated the presence of a myriad of abnormalities in mitochondrial membrane composition and cristae in FVB oocytes.

The most striking of these were the “onion-like” whorling of the peripheral cristae and the frequent sites of outer mitochondrial membrane rupture that allowed the inner boundary membranes to extend outward. A similar pathophysiology (i.e., onion-like mitochondria) has been observed in heart and muscle cells exposed to stress (Schwarz et al. 1998; Walker and Benzer 2004), and in yeast whose energy production is impaired (Paumard 2002), indicating that this phenotype is not unique to the germline of FVB mice. Moreover, the loss of cristae in mitochondria of yeast maintained under anoxic conditions is reversed by aeration (Lloyd et al. 2003), suggesting that a tremendous plasticity exists in mitochondrial ultrastructure and function. In agreement with this, both the structural anomalies in mitochondria and the deficiency in ROS production were reversed when FVB oocytes were incubated with pyruvate. These outcomes were paralleled by a complete absence of apoptosis in pyruvate-treated FVB oocytes during culture, suggesting the existence of a direct causal relationship between these mitochondrial defects and cellular susceptibility to apoptosis on this genetic background.

This conclusion is further supported by the finding that microinjection of FVB mitochondria into B6C3F I oocytes, which have an inherently low basal rate of death, increased the incidence of apoptosis in these cells by nearly 5-fold. These results indicated that the FVB mitochondria are supplying pro-apoptotic factors to the B6C3F1 oocytes, most likely by virtue of their “leaky” outer membrane structure. Consistent with this, mitochondria collected from FVB oocytes released approximately 20% more cytochrome c in short-term incubations than did mitochondria of B6C3F1 oocytes. However, direct microinjection of cytochrome c into B6C3F1 oocytes did not, by itself, induce apoptosis, suggesting that the FVB mitochondria must be supplying additional factors to trigger apoptosis. To this end, microinjection of recombinant Smac/DIABLO, a protein also released from mitochondria that suppresses activity of the caspase-blocking inhibitor-of-apoptosis proteins (Du et al. 2000; Verhagen et al. 2000), increased apoptosis in B6C3F1 oocytes. Furthermore, coinjecting cytochrome c synergistically enhanced this effect. Although Smac/DIABLO deficient mice were initially reported to have no discernible phenotype (Okada et al. 2002), outcrossing Smac/DIABLO deficient mice onto a FVB genetic background uncovered the functional importance of Smac/DIABLO to the increased susceptibility of FVB germ cells to apoptosis. Therefore, these findings, when considered with the other data discussed above, have collectively uncovered the basis for the strain-dependent enhancement of apoptosis in the FVB germline at the cellular, molecular and ultrastructural level.

In summary, increasing appreciation of the impact of background genetic variance on the ultimate phenotypic presentation of a Mendelian trait has been fueled by recent studies identifying specific loci that can enhance or suppress the onset of several genetically based diseases in mammals (Nadeau 2003). Additionally, as is the case with caspase-3 deficient mice discussed above, modifier genes can exert either repressive or synergistic effects on the emergence of a given phenotype in various gene mutant or transgenic mouse lines (Barthold 2004). Herein at least two different modifier loci have been identified that exert profound negative effects on the structural integrity of chromosomal DNA or mitochondria, both of which lead to a marked elevation in apoptosis susceptibility.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description. Such modifications are intended to fall within the scope of the appended claims.

All publications, patents and patent applications referred to herein arc incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methodologies etc. which are reported therein which might be used in connection with the invention. Nothing herein is to be construed as an admission that the invention is not entitled to antedate such disclosure by virtue of prior invention.

TABLE 1
Bcl-2 Family Proteins

| | | NCBI Accession No. of Nucleic Acid | Gene ID |
|---|---|---|---|
| Protein | NCBI Accession No. | Encoding Protein | No. |
| Bcl-2 | NP_000624, NP_000648, | NM_000633, NM_000657, | 596 |
| | AAO26045 | AC021803, AC022726, AY220759, | |
| | AAD14111, AAL02169, | S72602, AF401211, AI401297, | |
| | AAH27258 AAA51813, | BC027258, M13994 M13995, | |
| | AAA51814, AAA35591 | M14745, X06487 | |
| | CAA29778, P10415, Q96PA0 | | |
| Bcl-xL | NP_612815, NP_001182, | NM_138578, | 598 |
| Bcl-xS | CAI23025, CAI23026, CAI23027 | NM_001191, AL117381 | |
| | CAC10003, CAI12811, CAI12812 | AL160175, AL160175, U72398, | |
| | CAI12813, CAI12814, CAI12815 | AY263335, AY263336, BC019307 | |
| | CAC10003, CAI12811, CAI12812 | BT007208, BX647525, CR936637 | |
| | CAI12813, CAI12814, CAI12815 | Z23115, Z23116 | |
| | AAB17354, AAP22027, | | |
| | AAP22028 | | |
| | AAH19307, AAP35872, | | |
| | CAI56777, CAA80661, | | |
| | CAA80662, Q07817, | | |
| | Q5CZ89, Q5QP56, Q5QP59, | | |
| | Q5TE63, Q5TE64, Q5TE65 | | |
| | Q9H1R | | |
| Bcl-w | NP_004041, AAI04790 | NM_004050, BC104789 | 599 |
| | AAV38356, BAA19666, | BT019549, D87461 | |
| | AAB09055, Q92843 | U59747 | |
| Ced-9 | NP_499284, CAA82573 | NM_066883, Z29443 | 3565776 |
| | AAA20080, P41958 | L26545 | |
| Mcl-1 | NP_068779, NP_877495, | NM_021960, NM_182763, | 4170 |
| | AAF74821, AAG00896, | DQ088966, AA453505, AF118124 | |
| | AAF64255 | AF118276, AF118277, AF118278 | |
| | AAF64256, CAI15503, CAI15504 | AF203373, BC017197, BC071897 | |
| | AAY68220, AAD13299, | BC107735, BT006640, L08246, | |
| | AAF15309, AAF15310, | AF147742, AF162677, AF198614, | |
| | AAF15311 | AL356356, AA453505 | |
| | AAG00904, AAH17197, | | |
| | AAH71897 | | |
| | AAI07736, AAP35286, Q07820 | | |
| | Q9HD91, Q9UHR7, Q9UHR8 | | |
| | Q9UHR9, Q9UNJ1 | | |
| Aven | NP_065104, Q9NQS1, | NM_020371, AF283508 | 57099 |
| | AAF91470, AAH10488 | BC010488, BC063533 | |
| | AAH63533, Q9NQS1 | BU855198, CR618548, CR619789 | |
| Diva | NP_065129, CAD30221, | NM_020396, AJ458330 | 10017 |
| | AAG00503, AAK48715 | AF285092, AF326964 | |
| | AAH93826, AAH93828 | BC093826, BC093828 | |
| | AAI04443, AAI04444, Q52LQ9, | BC104442, BC104443 | |
| | Q9HD36 | | |

TABLE 2

| | | NCBI Accession No. of Nucleic Acid | Gene ID |
|---|---|---|---|
| Protein | NCBI Accession No. | Encoding Protein | No. |
| Rad51 | NP_002866, NP_597994, | NM_002875, NM_133487, | 5888 |
| Human | AAD49705, AAF61901, AAF69145, | AF165088 | |
| | AAN87149, BAD18467, AAR07948 | AF203691, AF233740, AY196785 | |
| | AAH01459, AAV38511, | AK131299, AY425955, BC001459 | |
| | CAG38796, BAA02962, BAA03189, | BT019705, CR536559, CR594665 | |
| | Q06609, Q5U0A5, Q6FHX9 | CR606487, CR619182, CR622784 | |
| | Q6TAR4, Q6ZNA8, Q9NZG9 | CR626167, D13804, D14134 | |
| Rad55 | NP_010361, AAU09690 | AY723773 | 851648 |
| Yeast | CAA98895 | Z74372 | |
| | BAA01284 | D10481 | |
| | CAA86798 | Z46796 | |
| | CAA57603, P38953 | X82086 | |
| Rad57 | NP_010287, CAA88064 | Z48008, M65061, | 851567 |
| Yeast | AAA34950, P25301 | | |
| Rad50 | NP_005723, NP_597816, | NM_005732, NM_133482, | 10111 |
| Human | AAD50325, AAD50326 | AF057299 | |
| | AAH62603, AAH73850 | AF057300 | |
| | AAI08283, AAB07119, | BC062603 | |
| | Q92878 | BC073850 | |
| | | BC108282 | |
| | | U63139 | |
| Rad52 | NP_002870, NP_602294, | NM_002879, NM_134422 | 5893 |
| Human | NP_602295, NP_602296, | NM_134423, NM_134424, | |
| | AAF05531, AAF05532 | AY527412 | |
| | AAF05533, AAF05534, AAS00097 | AF125948, AF125949, AF125950 | |
| | AAD24575, AAD24576, AAD24577 | AY540753, BC042136, BC104015, | |
| | AAT44403, AAI04016, AAI04017 | BC104016, BC104017, BC114954 | |
| | AAI04018, AAI14955, AAB05203, | L33262, U12134, U27516, | |
| | AAA85793, AAA87554, P43351, | AF187983 | |
| | Q5DR82, Q9UHE1, Q9UHE2 | | |
| | Q9UHE3, Q9UHE4, Q9Y5T7 | | |
| | Q9Y5T8, Q9Y5T9 | | |
| Rad54L | NP_003570, CAI22117, | NM_003579, AL121602 | 8438 |
| Human | AAT38113, CAA66379, Q5TE31 | AY623117, AA582917, BM464345 | |
| | Q92698 | CR591799, CR594300, X97795 | |
| Rad59 | NP_010224, AAB66660, | U53668, Z74107, AY693025 | 851500 |
| Yeast | CAA98622, AAT93044, Q12223 | | |
| Mre11 | NP_005581, NP_005582, | NM_005590, NM_005591, | 4361 |
| Human | AAK18790, AAS79320, AAD10197, | AF303395 | |
| | AAC36249, AAH05241, AAH63458, | AP000765, AY584241, AF022778, | |
| | AAP35376, AAC78721, P49959 | AF073362, AK095388, BC005241, | |
| | Q9BS79 | BC017823, BC063458, BF574168, | |
| | | BT006730, CR600174, U37359 | |
| XRS2 | NP_010657, AAB64805 | U28373 | 851975 |
| | AAA35220, P33301 | L22856 | |
| Lim15/ | NP_008999, Q14565 CAB45656, | NM_007068, AL022320 | 11144 |
| DMC1 | AAR89915, AAH35658, | AY520538, BC035658, | |
| | CAG30372, BAA09932, BAA10970 | BM545092 | |
| | | CR456486, D63882, D64108 | |

### Full Citations for References Referred to in the Specification

1. Adams J M, Cory S. Life-or-death decisions by the Bcl-2 protein family. Trends Biochem Sci 2001; 26: 61-66.

2. Green D R, Reed J C. Mitochondria and apoptosis. Science 1998; 281: 1309-1312.

3. Vaux D L, Korsmeyer S J. Cell death in development. Cell 1999; 96: 245-254.

4. Adams J M, Cory S. The Bcl-2 protein family: arbiters of cell survival. Science 1998; 281: 1322-1326.

5. Jurisicova A, Latham K E, Casper R F, Varmuza S L. Expression and regulation of genes associated with cell death during murine preimplantation embryo development. Mol Reprod Dev 1998; 51: 243-253.

6. Jurisicova A, Antenos M, Varmuza S, Tilly J L, Casper RF. Expression of apoptosis-related genes during human preimplantation embryo development: potential roles for the Harakiri gene product and Caspase-3 in blastomere fragmentation. Mol Hum Reprod 2003; 9: 133-141.

7. Jurisicova A, Perez G, Acton B, Casper R F, Tilly J. Oocyte survival is controlled by age related changes in death regulatory pathways. In: 49th Annual Meeting of Society for Gynecological Investigation; 2002; Los Angeles, Calif. 66A.

8. Jurisicova A, Casper, R F, Tilly, J. Bcl-x isoforms differentially affect preimplantation embryo development. In: Cold Spring Harbor Autum Symposia; 2001; Cold Spring Harbor.

9. Rucker E B, 3rd, Dierisseau P, Wagner K U, Garrett L, Wynshaw-Boris A, Flaws J A, Hennighausen L. Bcl-x and Bax regulate mouse primordial germ cell survival and apoptosis during embryogenesis. Mol Endocrinol 2000; 14: 1038-1052.

10. Riedlinger G, Okagaki R, Wagner K U, Rucker E B, 3rd, Oka T, Miyoshi K, Flaws J A, Hennighausen L. Bcl-x is not required for maintenance of follicles and corpus luteum in the postnatal mouse ovary. Biol Reprod 2002; 66: 438-444.

11. Rinkenberger J L, Horning S, Klocke B, Roth K, Korsmeyer S J. Mcl-1 deficiency results in peri-implantation embryonic lethality. Genes Dev 2000; 14: 23-27.

12. Kuwana T, Mackey M R, Perkins G, Ellisman M H, Latterich M, Schneiter R, Green D R, Newmeyer D D. Bid, Bax, and lipids cooperate to form supramolecular openings in the outer mitochondrial membrane. Cell 2002; 111: 331-342.

13. Steuerwald N, Cohen J, Herrera R J, Brenner C A. Analysis of gene expression in single oocytes and embryos by real-time rapid cycle fluorescence monitored RT-PCR. Mol Hum Reprod 1999; 5: 1034-1039.

14. Lewandoski M, Wassarman K M, Martin G R. Zp3-cre, a transgenic mouse line for the activation or inactivation of loxp-flanked target genes specifically in the female germ line. Curr Biol 1997; 7: 148-151.

15. Handyside A H, Hunter S. A rapid procedure for visualising the inner cell mass and trophectoderm nuclei of mouse blastocysts in situ using polynucleotide-specific fluorochromes. J Exp Zool 1984; 231: 429-434.

16. Hardy K, Handyside A H, Winston RM. The human blastocyst: cell number, death and allocation during late preimplantation development in vitro. Development 1989; 107: 597-604.

17. Hardy K. Cell death in the mammalian blastocyst. Mol Hum Reprod 1997; 3: 919-925.

18. Cummins JM. Mitochondria in reproduction. Reprod Biomed Online 2004; 8: 14-15.

19. Van Blerkom J. Mitochondria in human oogenesis and preimplantation embryogenesis: engines of metabolism, ionic regulation and developmental competence. Reproduction 2004; 128: 269-280.

20. Vander Heiden M G, Chandel N S, Schumacker P T, Thompson C B. Bcl-xL prevents cell death following growth factor withdrawal by facilitating mitochondrial ATP/ADP exchange. Mol Cell 1999; 3: 159-167.

21. Vander Heiden M G, Plas D R, Rathmell J C, Fox C J, Harris M H, Thompson C B. Growth factors can influence cell growth and survival through effects on glucose metabolism. Mol Cell Biol 2001; 21: 5899-5912.

22. Eliseev R A, Gunter K K, Gunter T E. Bcl-2 prevents abnormal mitochondrial proliferation during etoposide-induced apoptosis. Exp Cell Res 2003; 289: 275-281.

23. Piko L, Matsumoto L. Number of mitochondria and some properties of mitochondrial DNA in the mouse egg. Dev Biol 1976; 49: 1-10.

24. Jansen RP, de Boer K. The bottleneck: mitochondrial imperatives in oogenesis and ovarian follicular fate. Mol Cell Endocrinol 1998; 145: 81-88.

25. Dean N L, Battersby B J, Ao A, Gosden R G, Tan S L, Shoubridge E A. Prospect of preimplantation genetic diagnosis for heritable mitochondrial DNA diseases. Mol Hum Reprod 2003; 9: 631-638.

26. Vander Heiden M G, Choy J S, VanderWeele D J, Brace J L, Harris M H, Bauer D E, Prange B, Kron S J, Thompson C B, Rudin C M. Bcl-x(L) complements Saccharomyces cerevisiae genes that facilitate the switch from glycolytic to oxidative metabolism. J Biol Chem 2002; 277: 44870-44876.

27. Manfredi G, Kwong J Q, Oca-Cossio J A, Woischnik M, Gajewski C D, Martushova K, D'Aurelio M, Friedlich A L, Moraes C T. BCL-2 improves oxidative phosphorylation and modulates adenine nucleotide translocation in mitochondria of cells harboring mutant mtDNA. J Biol Chem 2003; 278: 5639-5645.

28. Martin K L, Leese H J. Role of glucose in mouse preimplantation embryo development. Mol Reprod Dev 1995; 40: 436-443.

29. Gardner D K, Leese H J. Non-invasive measurement of nutrient uptake by single cultured pre-implantation mouse embryos. Hum Reprod 1986; 1: 25-27.

30. Moley K H, Chi M M, Knudson C M, Korsmeyer S J, Mueckler M M. Hyperglycemia induces apoptosis in pre-implantation embryos through cell death effector pathways. Nat Med 1998; 4: 1421-1424.

31. Chi M M, Moley K H. Single-embryo measurement of basal- and insulin-stimulated glucose uptake. Methods Mol Med 2003; 83: 171-178.

32. Schmitt E, Paquet C, Beauchemin M, Bertrand R. Bcl-xES, a BH4- and BH2-containing antiapoptotic protein, delays Bax oligomer formation and binds Apaf-1, blocking procaspase-9 activation. Oncogene 2004; 23: 3915-3931.

33. Casper R F. Ovarian hyperstimulation: effects of GnRH analogues. Does triggering ovulation with gonadotrophin-releasing hormone analogue prevent severe ovarian hyperstimulation syndrome? Hum Reprod 1996; 11: 1144-1146.

34. Sun J G, Jurisicova A, Casper R F. Detection of deoxyribonucleic acid fragmentation in human sperm: correlation with fertilization in vitro. Biol Reprod 1997; 56: 602-607.

35. Grethe S, Ares M P, Andersson T, Porn-Ares M I. p38 MAPK mediates TNF-induced apoptosis in endothelial cells via phosphorylation and downregulation of Bcl-x(L). Exp Cell Res 2004; 298: 632-642.

36. Domina A M, Smith J H, Craig R W. Myeloid cell leukemia 1 is phosphorylated through two distinct pathways, one associated with extracellular signal-regulated kinase activation and the other with G2/M accumulation or protein phosphatase 1/2A inhibition. J Biol Chem 2000; 275: 21688-21694.

37. Domina A M, Vrana J A, Gregory M A, Hann S R, Craig R W. MCL1 is phosphorylated in the PEST region and stabilized upon ERK activation in viable cells, and at additional sites with cytotoxic okadaic acid or taxol. Oncogene 2004; 23: 5301-5315.

38. Clohessy J G, Zhuang J, Brady H J. Characterisation of Mcl-1 cleavage during apoptosis of haematopoietic cells. Br J Haematol 2004; 125: 655-665.

39. Michels J, O'Neill J W, Dallman C L, Mouzakiti A, Habens F, Brimmell M, Zhang K Y, Craig R W, Marcusson E G, Johnson P W, Packham G. Mcl-1 is required for Akata6 B-lymphoma cell survival and is converted to a cell death molecule by efficient caspase-mediated cleavage. Oncogene 2004; 23: 4818-4827.Acton, B. M., Jurisicova, A., Jurisica, I. and Casper, R. F. 2004. Alterations in mitochondrial membrane potential during preimplantation stages of mouse and human embryo development. Mol. Hum. Reprod. 10: 23-32.

40. Barthold, S. W. 2004. Genetically altered mice: phenotypes, no phenotypes, and Faux phenotypes. Genetica 122: 75-88.

41. Bernas, T., and Dobrucki, J. 2002. Mitochondrial and nonmitochondrial reduction of MTT: interaction of MTT with TMRE, JC-1, and NAO mitochondrial fluorescent probes. Cytometry 47: 236-242.

42. Bridges, C. B. 1919. Specific modifiers of eosin eye color in Drosophila melanogaster. J. Exp. Zool. 10: 337-384.

43. Charames, G. S., and Bapat, B. 2003. Genomic instability and cancer. Curr. Mol. Med. 3: 589-596.

44. Chipuk, J. E., Kuwana, T., Bouchier-Hayes, L., Droin, N. M., Newmeyer, D. D., Schuler, M., and Green, D. R. 2004. Direct activation of Bax by p53 mediates mitochondrial membrane permeabilization and apoptosis. Science 303: 1010-1014.

45. Danial, N. N., and Korsmeyer, S. J. 2004. Cell death: critical control points. Cell 116: 205-219.

46. Darley-Usmar, V. M., Rickwood, D., and Wilson, M. T. 1987. Isolation and characteristics of intact mitochondria. In Mitochondria, a practical approach. D. Rickwood and B. D. Hames, eds. (Oxford-Washington DC: IRL Press), pp 1-16.

47. Downs, S. M., and Hudson, E. D. 2000. Energy substrates and the completion of spontaneous meiotic maturation. Zygote 8: 339-351.

48. Dragani, T. A. 2003. 10 years of mouse cancer modifier loci: human relevance. Cancer Res. 63: 3011-3018.

49. Du, C., Fang, M., Li, Y., Li, L., and Wang, X. 2000. Smac, a mitochondrial protein that promotes cytochrome c-dependent caspase activation by eliminating IAP inhibition. Cell 102: 33-42.

50. Green, D. R., and Kroemer, G. 2004. The pathophysiology of mitochondrial cell death. Science 305: 626-629.

51. Jansen, R. P., and de Boer, K. 1998. The bottleneck: mitochondrial imperatives in oogenesis and ovarian follicular fate. Mol. Cell. Endocrinol. 145: 81-88.

52. Jiang, X., and Wang, X. 2004. Cytochrome C-mediated apoptosis. Annu. Rev. Biochem. 73: 87-106.

53. Karran, P. 2000. DNA double-strand break repair in mammalian cells. Curr. Opin. Genet. Dev. 10: 144-150.

54. Kroemer, G. 2003. Mitochondrial control of apoptosis: an introduction. Biochem. Biophys. Res. Commun. 304: 433-435.

55. Kuida, K., Zheng, T. S., Na, S., Kuan, C., Yang, D., Karasuyama, H., Rakic, P., and Flavell, R. A. 1996. Decreased apoptosis in the brain and premature lethality in CPP32-deficient mice. Nature 384: 368-372.

56. Kurumizaka, H., Aihara, H., Kagawa, W., Shibata, T., and Yokoyama S. 1999. Human Rad51 amino acid residues required for Rad52 binding. J. Mol. Biol. 291: 537-548.

57. Leonard, J. R., Klocke, B. J., D'Sa, C., Flavell, R. A., and Roth, K. A. 2002. Strain-dependent neurodevelopmental abnormalities in caspase-3-deficient mice. J. Neuropathol. Exp. Neurol. 61: 673-677.

58. Li, L., and Zou, L. 2005. Sensing, signaling, and responding to DNA damage: organization of the checkpoint pathways in mammalian cells. J. Cell. Biochem. 94: 298-306.

59. Liu, X., Kim, C. N., Yang, J., Jemmerson, R., and Wang, X. 1996. Induction of apoptotic program in cell-free extracts: requirement for dATP and cytochrome c. Cell 86: 147-157.

60. Lloyd, D., Lemar, K. M., Salgado, L. E., Gould, T. M., and Murray, D. B. 2003. Respiratory oscillations in yeast: mitochondrial reactive oxygen species, apoptosis and time; a hypothesis. FEMS Yeast Res. 3: 333-339.

61. Lombard, D. B., Chua, K. F., Mostoslavsky, R., Franco, S., Gostissa, M., and Alt, F. W. 2005. DNA repair, genome stability, and aging. Cell 120: 497-512.

62. Matikainen, T., Perez, G. I., Zheng, T. S., Kluzak, T. R., Rueda, B R., Flavell, R. A., and Tilly, J. L. 2001. Caspase-3 gene knockout defines cell lineage specificity for programmed cell death signaling in the ovary. Endocrinology 142: 2468-2480.

63. Mastronarde, D. N. 1997. Dual-axis tomography: an approach with alignment methods that preserve resolution. J. Struct. Biol. 120: 343-352.

64. Morita, Y., Perez, G. I., Paris, F., Miranda, S. R., Ehleiter, D., Haimovitz-Friedman, A., Fuks, Z., Xie, Z., Reed, J. C., Schuchman, E. H., Kolesnick, R. N., and Tilly, J. L. 2000. Oocyte apoptosis is suppressed by disruption of the acid sphingomyelinase gene or by sphingosine-1-phosphate. Nat. Med. 6: 1109-1114.

65. Nadeau, J. H. 2003. Modifier genes and protective alleles in humans and mice. Curr. Opin. Genet. Dev. 13: 290-295.

66. Nasr-Esfahani, M. H., and Johnson, M. H. 1992. Quantitative analysis of cellular glutathione in early preimplantation mouse embryos developing in vivo and in vitro. Hum. Reprod. 7: 1281-1290.

67. Okada, H., Suh, W.-K., Jin, J., Woo, M., Du, C., Elia, A., Duncan, G. S., Wakeham, A., Itie, A., Lowe, S. W., Wang, X., and Mak, T. W. 2002. Generation and characterization of Smac/DIABLO-deficient mice. Mol. Cell. Biol. 22: 3509-3517.

68. Paumard P, Vaillier J, Coulary B, Schaeffer J, Soubannier V, Mueller D M, Brethes D, di Rago J P, and Velours J. 2002. The ATP synthase is involved in generating mitochondrial cristae morphology. EMBO J. 21: 221-230.

69. Perez, G. 1, Knudson, C. M., Leykin, L., Korsmeyer, S. J., and Tilly, J. L. 1997. Apoptosis-associated signaling pathways are required for chemotherapy-mediated female germ cell destruction. Nat. Med. 3: 1228-1332.

70. Perez, G. 1, Tao, X. J., and Tilly, J. L. 1999. Fragmentation and death (a.k.a. apoptosis) of ovulated oocytes. Mol. Hum. Reprod. 5: 414-420.

71. Perkins, G., Renken, C., Martone, M., Young, S., Ellisman, M. H., and Frey, T. 1997a, Electron tomography of neuronal mitochondria: 3-D structure and organization of cristae and membrane contacts. J. Struct. Biol. 119: 260-272.

72. Perkins, G., Renken, C., Song, J. Y., Frey, T. G., Young, S., Lamont, S., Martone, M., Lindsey, S., Frey, T., and Ellisman, M. 1997b. Electron tomography of large, multicomponent biological structures. J. Struct. Biol. 120: 219-227.

73. Perkins, G. A., Renken, C. W., Frey, T. G., and Ellisman, M. H. 2001. Membrane architecture of mitochondria in neurons of the central nervous system. J. Neurosci. Res. 66: 857-865.

74. Perkins, G. A., Ellisman, M. H., and Fox, D. A. 2003. Three-dimensional analysis of mouse rod and cone mitochondrial cristae architecture: bioenergetic and functional implications. Mol. Vision 9: 60-73.

75. Riazuddin, S., Castelein, C. M., Ahmed, Z. M., Lalwani, A. K., Mastroianni, M. A., Naz, S., Smith, T. N., Liburd, N. A., Friedman, T. B., Griffith, A. J., et al. 2000. Dominant modifier DFNM1 suppresses recessive deafness DFNB26. Nat. Genet. 26: 431-434.

76. Ricci, J. E., Munoz-Pinedo, C., Fitzgerald, P., Bailly-Maitre, B., Perkins, G. A., Yadava, N., Scheffler, I. E., Ellisman, M. H., and Green, D. R. 2004. Disruption of mitochondrial function during apoptosis is mediated by caspase cleavage of the p75 subunit of complex I of the electron transport chain. Cell 117: 773-786.

77. Salemme, F. R. 1977. Structure and function of cytochrome c. Annu. Rev. Biochem. 46: 299-329.

78. Schwarz, E. R., Pollick, C., Dow, J., Patterson, M., Birnbaum, Y., and Kloner, R. A. 1998. A small animal model of non-ischemic cardiomyopathy and its evaluation by transthoracic echocardiography. Cardiovasc. Res. 39: 216-223

79. Slavotinek, A., and Biesecker, L. G. 2003. Genetics modifiers in human development and malformation syndromes, including chaperone proteins. Hum. Mol. Genet. 12 (Special No. 1): R45-R50.

80. Sutovsky, P. 2004. Degradation of paternal mitochondria after fertilization: implications for heteroplasmy, assisted reproductive technologies and mtDNA inheritance. Reprod. Biomed. Online 8: 24-33.

81. Taylor, R. W., and Turnbull, D. M. 2005. Mitochondrial DNA mutations in human disease. Nat. Rev. Genet. 6: 389-402.

82. Thacker, J. 2005. The RAD51 gene family, genetic instability and cancer. Cancer Lett. 219: 125-135.

83. Tilly, J. L. 2001. Commuting the death sentence: how oocytes strive to survive. Nat. Rev. Mol. Cell. Biol. 2: 838-848.

84. Verhagen, A. M., Ekert, P. G., Pakusch, M., Silke, J., Connolly, L. M., Reid, G. E., Moritz, R. L., Simpson, R. J., and Vaux, D. L. 2000. Identification of DIABLO, a mammalian protein that promotes apoptosis by binding to and antagonizing IAP proteins. Cell 102: 43-53.

85. Walker, D. W., and Benzer, S. 2004. Mitochondrial “swirls” induced by oxygen stress and in the Drosophila mutant hyperswirl. Proc. Natl. Acad. Sci. USA 101: 10290-10295.

86. 48. Wallace, D. C. 2005. A mitochondrial paradigm of metabolic and degenerative diseases, aging and cancer: a dawn for evolutionary medicine. Annu. Rev. Genet. July 19 Epub ahead of print.

87. Yang, H. W., Hwang, K. J., Kwon, H. C., Kim, H. S., Choi, K. W., and Oh, K. S. 1998. Detection of reactive oxygen species (ROS) and apoptosis in human fragmented embryos. Hum. Reprod. 13: 998-1002.

88. Zielenski, J., Corey, M., Rozmahel, R., Markiewicz, D., Aznarez, I., Casals, T., Larriba, S., Mercier, B., Cutting, G. R., Krebsova, A., et al. 1999. Detection of a cystic fibrosis modifier locus for meconium ileus on human chromosome 19q13. Nat. Genet. 22, 128-129.

89. Benson, F. E., A Stasiak, and S C West. 1994, Purification and characterization of the human Rad51 protein, an analogue of E. coli RecA. EMBO J. 13(23): 5764-5771.

90. Chi M M, Hoehn A, Moley K H. Metabolic changes in the glucose-induced apoptotic blastocyst suggest alterations in mitochondrial physiology. Am J Physiol Endocrinol Metab 2002; 283: E226-232.

1. A method for enhancing developmental potential of oocytes or preimplantation embryos, improving the success of in vitro fertilization, improving the success of gamete intrafallopian transfer, or improving the success of zygote intrafallopian transfer, said method comprising modulating one or more mitochondrial-associated proteins, genomic integrity modifier proteins, or a combination thereof in the oocytes or preimplantation embryos.

2. (canceled)

3. A method according to claim 1, wherein the levels of one or more mitochondrial-associated proteins, genomic integrity modifier proteins, or a combination thereof are increased by introducing the proteins into the oocytes or preimplantation embryos.

4. A method according to claim 3 wherein the proteins are introduced by microinjection or electrofusion.

5-6. (canceled)

7. A method according to claim 1, further comprising fertilizing the oocyte to obtain a zygote with increased levels of mitochondrial-associated proteins, genomic integrity modifier proteins, or a combination thereof.

8. A method for fertilizing oocytes, said method comprising removing oocytes from a follicle of an ovary, modulating one or more mitochondrial-associated proteins, genomic integrity modifier proteins, or a combination thereof, and fertilizing the resulting oocytes with spermatozoa.

9. A method according to claim 8, wherein the mitochondrial-associated proteins, genomic integrity modifier proteins. or a combination thereof are introduced into the oocytes.

10. A method of according to claim 8 wherein modulating the proteins and fertilizing the spermatozoa can be carried out simultaneously, sequentially, or separately.

11. A method for improving embryo development after in vitro fertilization or embryo transfer in a female mammal comprising implanting into the female mammal an embryo derived from an oocyte or preimplantation embryo wherein one or more mitochondrial-associated proteins, genomic integrity modifier proteins, or a combination thereof have been modulated.

12. A method according to claim 11, wherein the oocyte or preimplantation embryo comprises increased levels of one or more pro-survival Bcl-2 family proteins, and/or Rad51 family proteins, or a combination thereof.

13. (canceled)

14. A method according to claim 1 which is for improving the success of in vitro fertilization in a female subject and comprises:
(a) removing oocytes from the subject;
(b) modulating one or more mitochondrial-associated proteins, genomic integrity modifier proteins, or a combination thereof in the oocytes;
(c) fertilizing the oocytes with spermatozoa; and
(d) transferring fertilized oocytes from step (c) into the uterus of the subject.

15. A method for enhancing developmental potential of recipient oocytes in a nuclear transfer method, said method for enhancing developmental potential of recipient oocytes comprising introducing one or more mitochondrial-associated proteins, and/or genomic integrity modifier proteins or a combination thereof into the recipient oocytes.

16. (canceled)

17. A method according to claim 15 wherein the mitochondrial-associated Bcl 2 family protein is a pro-survival Bcl-2 family protein.

18. A method according to claim 17, wherein the pro-survival Bcl-2 family protein is Bcl-2, Bcl-xL, Mcl-1, Diva, or Aven.

19. A method according to preceding claim 1, wherein the genomic integrity modifier protein is a RecA family protein or a Rad51 family protein.

20. (canceled)

21. A method according to claim 19, wherein the genomic integrity modifier protein is Rad51.

22-27. (canceled)

28. A composition for enhancing developmental potential of oocytes and preimplantation embryos, said composition comprising one or more mitochondrial-associated proteins genomic integrity modifier proteins, or a combination thereof and a pharmaceutically acceptable carrier, excipient or diluent.

29. (canceled)

30. A composition according to claim 28, wherein the mitochondrial-associated protein is a pro-survival Bcl-2 family protein.

31. A composition according to any preceding claim 28, wherein the genomic integrity modifier protein is a RecA family protein or a Rad51 family protein.

32. (canceled)

33. A composition according to claim 31, wherein the genomic integrity modifier protein is Rad51.

34. A kit for carrying out a method of claim 1.

35. (canceled)
